(19) United States
(12) Patent Application Publication
(10) Pub. No.: US 2023/0220065 A1
(43) Pub. Date: Jul. 13, 2023

(54) ANTIBODIES TO INTERLEUKIN-1BETA AND USES THEREOF

(71) Applicant: ACADEMIA SINICA, Taipei (TW)

(72) Inventors: Andrew H.-J. WANG, San Diego, CA (US); Wen-Chih KUO, Taipei (TW); Cheng-Chung LEE, Taipei (TW); An-Suei YANG, Taipei (TW)

(73) Assignee: ACADEMIA SINICA, Taipei (TW)

(21) Appl. No.: 18/010,647

(22) PCT Filed: Jun. 15, 2021
(86) PCT No.: PCT/US2021/037390

§ 371 Date: Dec. 15, 2022

Related U.S. Application Data
(60) Provisional application No. 63/039,680, filed on Jun. 16, 2020.

(51) Int. Cl. C07K 16/24; A61P 35/00; A61K 47/26; C12N 15/63

(52) U.S. Cl. CPC C07K 16/245; A61P 35/00; A61K 47/26; C12N 15/63; A61K 2039/505

(57) ABSTRACT

Disclosed herein are anti-IL-1β antibodies capable of binding to human IL-1β and blocking its biological activities. Also provided herein are pharmaceutical compositions comprising the anti-IL-1β antibodies and therapeutic and diagnostic uses of such antibodies.

### CROSS-REFERENCE TO RELATED APPLICATION

This application claims the benefit of U.S. Provisional Application No. 63/039,680, filed Jun. 16, 2020, which is hereby incorporated by reference herein for all purposes.

### BACKGROUND OF INVENTION

Interleukin-1β (IL-1β), a member of the interleukin-1 family and a potent pleiotropic cytokine, plays a central role in protecting cells from microbial pathogen infections and endogenous stress stimuli. IL-1β is mainly released by monocytes, tissue macrophages, and dendritic cells in response to infection or injury. It also affects other immune cells (Th17 differentiation and B-cell proliferation in an IL-6-dependent manner). IL-1β binds to two cellular receptors, IL-1RI and IL-1RII. IL-1RI transduces the functional activity of IL-1β, but IL-1RII is considered to be a decoy receptor that negatively regulates IL-1β activity due to the lack of a C-terminal tail to transduce the signal. After IL-1β binds to IL-1RI and recruits the coreceptor chain, IL-1 receptor accessory protein (IL-1RAcP), the signaling culminates to activate NF-κB (FIG. 1A). While IL-1β initiates the reaction, the body also has a reverse signal to tightly regulate the amplification reaction produced by IL-1β. Except for IL-1RII, IL-1Ra is a naturally occurring IL-1 receptor antagonist since it has 30% amino acid sequence homology to IL-1β and binds to human type I and II IL-1 receptors without apparent cellular activation.

Many pathophysiological diseases have been attributed to the derailment of IL-1β regulation, including hereditary autoinflammatory diseases (cryopyrin-associated periodic syndrome and neonatal-onset multisystem inflammatory disease) and complex chronic diseases (gout, type II diabetes mellitus, amyotrophic lateral sclerosis and so on). There are two possible mechanisms used in clinical practice for biologics-mediated IL-1β blockade: (1) reagents that block the binding of IL-1β to IL-1R, and these include anakinra and canakinumab; and (2) antibodies that inhibit the recruitment of IL-1RAcP but not the binding of IL-1β to IL-1R, and this includes gevokizumab. In recent years, the IL-1β blockade strategy has been extended to many other indications, such as cancer, type II diabetes, and rheumatoid arthritis. However, these drugs are not clinically effective for every patient. The reason may be that these biological agents could not fully inhibit the inflammatory response. Having more than one clinical drug is important to meet the potential needs of the physician, patient tolerability, pharmacoeconomic impact of global health and health-related quality of life. It is therefore of great interest to develop new antagonists for use in treating diseases associated with the IL-1β signaling.

### SUMMARY OF INVENTION

For this reason, we developed antibodies with new binding epitopes and high binding capacity, which is expected to remedy the shortcoming that other drugs cannot completely block IL-1β-induced signaling. The IL-1β-specific antibody, IgG26AW, was developed by screening a GH2 synthetic human phage-displayed library and constructed from a structure-based design. IgG26AW was characterized by in vitro biophysical and cell-based functional assays using either recombinant or naturally produced mature IL-1β protein from bacteria or human THP-1 cells (data not shown). In this report, we also validated IgG26AW-neutralizing antibodies specific for IL-1β in vivo to prevent human IL-1β-induced IL-6 elevation in C56BL/6 JNarl mice. IgG26AW had a higher inhibitory power for IL-1β than the marketed product canakinumab and significantly reduced clinical inflammation both in cell-based functional assays and mouse models. The cancer treatment of IgG26AW in A549 and MDA-MB-231 xenograft mouse models also made tumors shrink and inhibited tumor metastasis. These data indicated that IL-1β blockade by IgG26AW has high potential for therapeutic antibody development.

Moreover, IgG26AW neutralizes IL-1β 's biological activity by blocking the binding of IL-1β to the cell surface receptor IL-1R and its association with IL-1RAcP, thereby preventing the initiation of downstream intracellular signaling by the receptor. This competition mechanism of IgG26AW was visualized by analyzing the 26-Fab/IL-1β crystallography structure. The 26-Fab shows a large overlapping region with IL-1RI, as well as a small overlapping region with IL-1RAcP. This result indicated that IgG26 binding to IL-1β blocks interactions with both IL-1RI and IL-1RAcP simultaneously to prevent IL-1β-induced ternary complex formation. In conclusion, IgG26AW was selected from a generic human phage-display library, evolved through structural analysis, and showed superior neutralization activity due to its optimal binding with IL-1β. Our antibody provides new avenues for the treatment of cancer and other inflammatory-related diseases.

Thus, the present disclosure, at least in part, is based on the development of anti-IL-1β antibodies, e.g., IgG26AW and its variants, which showed high binding affinity and specificity to human IL-1β, and potent activities in inhibiting IL-1β induced cell proliferation and cytokine production (e.g., IL-6).

Accordingly, one aspect of the present disclosure relates to an isolated anti-IL-1β antibody that bind to human IL-1β (anti-IL-1β antibodies). The anti-IL-1β antibody disclosed herein may comprise a heavy chain variable domain (V_{H}), which comprises:

(i) a heavy chain complementary determining region 2 (HC CDR2) set forth as WPX₁X₂GX₃TY or WPX₁GX₃TY, in which X₁, X₂ or X₃ is selected from any one of amino acids, and

(ii) a heavy chain complementary determining region 3 (HC CDR3) comprising NGYWNYI, AGHHTGA, ALKPTSA, DSRKPRAM, GPGHTNA, or ETNPIQA.

The anti-IL-1β antibody disclosed herein, may comprise a light chain variable domain (V_{L}), which comprises:

(i) a light chain complementary determining region 1 (LC CDR1) set forth as X₄X₅G, in which X₄ or X₅ is selected from any one of amino acids, and

(ii) a light chain complementary determining region 3 (LC CDR3) comprising YSNFPI.

In an embodiment, the anti-IL-1β antibody disclosed herein may comprise a heavy chain variable domain (V_{H}), which comprises:

(i) a heavy chain complementary determining region 2 (HC CDR2) set forth as WPX₁X₂GX₃TY, in which X₁ is selected from the group of amino acids consisting of Y and R, X₂ is selected from the group of amino acids consisting of G and E, and X₃ is selected from the group of amino acids consisting of F and W, and

(ii) a heavy chain complementary determining region 3 (HC CDR3) selected from the group of amino acids consisting of NGYWNYI, AGHHTGA, ALKPTSA, DSRKPRAM, GPGHTNA, and ETNPIQA.

In an embodiment, the anti-IL-1β antibody disclosed herein, may comprise a light chain variable domain (V_{L}), which comprises:

(i) a light chain complementary determining region 1 (LC CDR1) set forth as X₄X₅G, in which X₄ is selected from the group of amino acids consisting of S, A, and R, and X₅ is selected from the group of amino acids consisting of W, G, and Q, and

(ii) a light chain complementary determining region 3 (LC CDR3) set forth YSNFPI.

In some embodiments, the anti-IL-1β antibody disclosed herein may further comprise a heavy chain complementary determining region 1 (HC CDR1) comprising VDMA, KDNA, KDMA, DHNA, SHMA, DNAA, or NGYS. Preferably, the anti-IL-1β antibody disclosed herein may further comprise a heavy chain complementary determining region 1 (HC CDR1) selected from the group of amino acids consisting of VDMA, KDNA, KDMA, DHNA, SHMA, DNAA, and NGYS. In some embodiments, the anti-IL-1β antibody disclosed herein may further comprise a light chain complementary determining region 2 (LC CDR2) comprising YSTAS, SQSTD, or HTSRS. Preferably, the anti-IL-1β antibody disclosed herein may further comprise a light chain complementary determining region 2 (LC CDR2) selected from the group of amino acids consisting of YSTAS, SQSTD, and HTSRS.

In some embodiments, the isolated antibody comprises the same HC CDRs and LC CDRs as a reference anti-IL-1β antibody, e.g. IgG26AW. In some examples, the isolated antibody disclosed herein, may comprises a
V_{H} comprising the amino acid sequence of:

EVQLVESGGGLVQPGGSLRLSCAASGFTIVDMAIHWVRQAPGKGLEWVAR
IWPREGWTYYADSVKGRFTISADTSKNTAYLQMNSLRAEDTAVYYCARFN
GYWNYIMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKAEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKDYKCKVSNKALPAPIEKTISKAKGQPREPQ
VYTLPPSRDELTRNQVSLTCLVKGFYPSDIAVEWESNGQPENNYKTTPPV
LDSDGSFFLYSKLTVDKSRWQQGNVFSCSVMHEALHNHYTQKSLSLSPG
K;

and/or
a V_{L} comprising the amino acid sequence of:

DIQMTQSPSSLSASVGDRVTITCRASQDVSWGVAWYQQKPGKAPKLLIHT
SRSLYSGVPSRFSGSGSGTDFTLTISSLQPEDFATYYCQQYSNFPITFGD
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

Any of the antibodies disclosed herein may specifically bind human IL-1β. Alternatively, the antibody may cross react with a non-human IL-1β, for example, a IL-1β of a non-human primate (e.g., Rhesus macaque).

Further, the instant disclosure provides an isolated antibody, which binds to the same epitope as antibody IgG26AW. In some examples, the isolated antibody disclosed herein, may comprise a HC CDR1, a HC CDR2, and a HC CDR3, which collectively contains no more than 10 amino acid variations, preferably no more than 8 amino acid variations, and more preferably no more than 5, 4, 3, 2 or 1 amino acid variations, as compared with the HC CDR1, HC CDR2, and HC CDR3 of antibody IgG26AW. The IL-1β antibody disclosed herein may further comprises an LC CDR1, an LC CDR2, and an LC CDR3, which collectively contains no more than 10 amino acid variations, preferably no more than 8 amino acids variations, and more preferably no more than 5, 4, 3, 2 or 1 amino acid variations, as compared with the LC CDR1, LC CDR2 and LC CDR3 of antibody IgG26AW.

Alternatively or in addition, the isolated antibody disclosed herein, may comprise heavy chain variable domain (V_{H}) that is at least 80% identical to the heavy chain variable domain of antibody IgG26AW, and a light chain variable domain (V_{L}) that is at least 80% identical to the light chain variable domain of antibody IgG26AW.

Any of the isolated antibody disclosed herein may be a human antibody or a humanized antibody. In some examples, any of the anti-IL-1β antibody described herein may be a full-length antibody (e.g., an IgG molecule). Alternatively, the anti-IL-1β antibody may be an antigen-binding fragment thereof.

Any of the anti-IL-1β antibodies disclosed herein may be conjugated with a detectable label.

In another aspect, provided herein is a nucleic acid or a nucleic acid set, which collectively encode the antibody binding to any of the IL-1β antibodies described herein. A nucleic acid set refers to two nucleic acid molecules one encoding the heavy chain and the other encoding the light chain of a multi-chain IL-1β antibody disclosed herein. In some examples, the nucleic acid or nucleic acid set can be a vector or a vector set, for example, an expression vector or an expression vector set. Also provide herein are host cells comprising the vector or vector set disclosed herein. Such host cells can be bacterial cells, yeast cells, insect cells, plant cells, or mammalian cells.

In addition, the present disclosure features a pharmaceutical composition, comprising (a) a monoclonal antibody binding or antigen binding fragments to IL-1β as disclosed herein, or the encoding nucleic acid(s), and (b) a pharmaceutically acceptable carrier. In some embodiments, the pharmaceutical acceptable carrier may comprise a buffering agent, a surfactant, a salt, an amino acid, an antioxidant, a sugar derivative (e.g., a non-reducing sugar, a sugar alcohol, a polyol, a disaccharide, or a polysaccharide). Such a pharmaceutical composition can be used for treating any of the target diseases also disclosed herein. Further, the present disclosure provides uses of the antibodies, the encoding nucleic acids, or other aspects relating to the antibody as disclosed herein for manufacturing a medicament for use in treatment of the target disease. In some examples, the pharmaceutical composition may further comprise 1,2,3,4,6-Penta-O-Galloyl-β-D-Glucose (PGG). In an example, a concentration of the PGG ranges from 1-500 μM. In another example, a concentration of the isolated antibody ranges from 1-1000 pM.

Further, the present disclosure features a method for treating IL-1β mediated disease in a subject, the method comprising administering to a subject in need thereof an effective amount of the anti-IL-1β antibody, or pharmaceutical composition comprising such. In some examples, the subject is a human patient having, suspected of having, or at risk for a disease, which is an inflammatory disease, an autoimmune disease or cancer. In some examples, the IL-1β mediated disease may be gout, type II diabetes mellitus, or amyotrophic lateral sclerosis.

Exemplary autoimmune diseases include, but are not limited to, cryopyrin-associated periodic syndrome, neonatal-onset multisystem inflammatory disease, rheumatoid arthritis, juvenile rheumatoid arthritis, spondyloarthropathy, ankylosing spondylitis, multiple sclerosis, psoriasis, plaque psoriasis, acute gouty arthritis, or osteoarthritis.

Exemplary inflammatory diseases include, but are not limited to, Kawasaki disease, chimeric antigen receptor T cell (CAR-T) induced cytokine release syndrome, CAR-T-induced related encephalopathy, diffuse parenchymal lung disease (DPLD), chronic obstructive pulmonary disease (COPD), aortic aneurysm, neuropathic pain, or graft-versus-host disease (GVHD).

Exemplary cancers include, but are not limited to, leukemia, gastric carcinoma, adenocarcinoma, mesothelioma, lung cancer, breast cancer, prostate cancer, colon cancer, head and neck cancer, melanoma, pancreatic ductal adenocarcinoma, colorectal cancer (CAC, for example, colitis-associated), or hypereosinophilic syndrome (HES). In some examples, the leukemia can be juvenile myelomonocyte leukemia (JMML), chronic myelomonocytic leukemia (CMML) or chronic eosinophilic leukemia.

In any of the methods disclosed herein, the subject has undergone or is undergoing an additional treatment of the disease.

Further, the present disclosure provides a method for producing an antibody binding to human IL-1β, the method comprising: (i) culturing the host cell of expressing the anti-IL-1β antibodies as disclosed herein under conditions allowing for expressing of the antibody that binds human IL-1β; and (ii) harvesting the cultured host cell or culture medium for collection of the antibody that binds human IL-1β. The method may further comprise (iii) purifying the antibody that binds human IL-1β.

In addition, the present disclosure also provides a method for detecting presence of IL-1β, the method comprising (i) contacting a biological sample suspected of containing IL-1β with the antibodies disclosed herein, and (ii) measuring binding of the antibody to IL-1β in the sample. The biological samples may be obtained from a human subject suspect of having or at risk of for a disease associated with IL-1β. The contact step may be performed by administering the subject an effective amount of the anti-IL-1β antibody.

The details of one or more embodiments of the invention are set forth in the description below. Other features or advantages of the present invention will be apparent from the following drawing and detailed description of several embodiments, and also from the appended claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates inhibitory effect on IL-1β-induced NF-κB signaling of different selected IgGs from generic human phage-display libraries in HEK-blue IL-1β cells. (A) IL-1β bound to its receptor IL-1RI and its receptor accessory protein IL-1RAcP to form the IL-1β/IL-1RI/IL-1RAcP ternary complex. IL-1β induces downstream signaling pathways, including NF-κB activation (IκB degradation) and AP-1 activation (phosphorylation of JNK, p38 and ERK). (B) Ten IgGs bound to human IL-1β were selected from the generic human phage-display library. The y-axis of the plots shows the percentage of the remaining NF-κB signaling after IgG treatments in 30 pM IL-1β treated HEK-blue IL-1β cells; the x-axis of the plots shows the number of treated IgGs. Only IgG26 showed an inhibitory effect at a high IgG concentration (69 nM).

FIG. 2 illustrates the IgG26 epitope mapping by X-ray crystallography. (A) Stereoview of the IL-1β/26-Fab complex structure. IL-1β is shown as a purple ribbon. The heavy chain and light chain of Fab are shown as light blue and green ribbons, respectively. (B) IL-1β/26-Fab interaction interface. The epitope on IL-1β is shown as a purple ribbon, and the stick side chain models with the surface that contains protrusions and concave cavities for amino acids are packed with the CDRs of 26-Fab. The heavy-chain and light-chain CDRs are shown as ribbons, and the contacting residues are shown as stick models with light blue and green C atoms, respectively. (C) (D) Comparison of 26-Fab and 26AW-Fab for IL-1β binding. The mutated residues of 26AW-Fab H-CDR2 are indicated as orange stick models.

FIG. 3 illustrates IL-1β signaling inhibition mechanism and interacting residues with IL-1β of IgG26. (A) (B) Top and side views of the IL-1β signaling complex blocked by antibodies. The IL-1β (purple surface) forms the signaling complex with IL-1RI and IL-1RAcP; IL-1RI and IL-1RAcP are shown with a translucent surface. The IL-1β/Fab complex of gevokizumab (yellow), canakinumab (blue) and 26-Fab (orange) are superimposed on the IL-1β signaling complex. (C) The amino acid comparison between human and mouse active IL-1β sequences. The gray square indicates the IL-1RI binding residues on IL-1β. The hallow squares indicate that the accessory binding protein interacting sites on IL-1β. The diamond indicates the residues on IL-1β that interacted with IgG26.

FIG. 4 illustrates the comparison of IL-1β neutralizing abilities in different IL-1β-neutralizing antibodies. IL-1β was diluted from 1500 to 0 pM. Each antibody was added at a 5 nM concentration with different concentrations of IL-1β. HEK-blue-IL-1β cells were used to detect the IgG inhibitory effects of IL-1β signaling. The binding curve was fitted by a nonlinear regression curve to obtain the IC50. IgG26AW can inhibit half of the NF-κB signal produced by 1.177 nM IL-1β. Compared together, gevokizumab and canakinumab can only inhibit half of the NF-κB signal produced by 0.3526 nM and 0.9254 nM IL-1β, respectively.

FIG. 5 illustrates cytokine biomarker assay. (A) The concentration of injected IgG1 at different time courses to compare the stability of IgG in male C57BL/6 mouse serum. IgG26AW and canakinumab have similar stabilities in mouse serum. (B) Male C57BL/6 mice were pretreated intravenously with neutralizing antibody (canakinumab, IgG26AW, and isotype IgG) at 0.2 mg/kg. Then, the mice were injected intraperitoneally with human IL-1β (240 ng/200 μL/mouse). Two hours after the injection, the mice were restrained, and blood was collected to detect the serum IL-6 level in the mice. IgG26AW treatment can inhibit the 40% induction of IL-6, which responds to human IL-1β boosting.

FIG. 6 illustrates the effects of IgG26AW in lung cancer A549 cells and an A549 xenograft nude mouse model. (A) Recombinant IL-1β induces strong NF-κB signaling in A549 cells. The treatment of IgG26AW reduced p-JNK and p-p38 phosphorylation signals and IκB-α degradation, which was induced by different dosages of IL-1β (0-1000 pM). (B) Treatment of A549 tumor-bearing nude mice with IgG26AW (10 μg/kg) reduced the tumor size compared to that with isotype IgG. (C) The changes in body weight of the IgG-treated nude mice are not different between the groups.

FIG. 7 illustrates the effects of IgG26AW in the MDA-MB-231 orthotopic ASID mouse breast cancer model. (A) Orthotopic tumors were harvested and compared after 5 weeks of IgG26AW treatment. (B) The records of tumor growth 6 weeks after treatment with IgG26AW. (C) The body weight changes in IgG-treated ASID mice. (D) The percentage of mice with tumor/metastasis from the mammary fat pad in each individual organ. These data have shown the inhibitory effects of IgG26AW on tumor growth and tumor metastasis.

FIG. 8 illustrates binding affinities and inhibitory effects of IgG variants selected from optimized IgG26 phage-display libraries. (A) ELISA assays demonstrated that the binding affinities of optimized clones, except H3-4, against IL-1β were much better than that of the original IgG26. The EC50 of each IgG is shown in Table 3. (B) Inhibitory effects on IL-1β-mediated downstream signaling were examined in 30 pM IL-1β-stimulated HEK-blue reporter cells. CDR H1-1 and CDR L2-2 clones showed the strongest suppression effects in a dose-dependent manner.

FIG. 9 illustrates IL-1β/26A-Fab complex structure. (A) The complex structure of IL-1β/26A-Fab (orange) is superimposed onto the IL-1β/26-Fab complex structure (green). (B) The 26A-Fab interacts with IL-1β. IL-1β is shown as a purple ribbon with a gray surface. The 26A-Fab is shown as a light blue ribbon. The key residues for this interaction are shown as stick models. The mutated residues Y54R and G55E in H-CDR2 of 26A-Fab are indicated as orange stick models.

FIG. 10 illustrates the binding kinetics of each IgG against IL-1β were measured by SPR. Binding kinetics of IgG26 (A), IgGF4 (B), IgG26A (C), and IgG26AW (D) for IL-1β were estimated by Biacore T100. Antibodies were injected on the biosensor surface immobilized with an anti-human IgG-specific antibody, and recombinant IL-1β was diluted and injected at six concentrations (2.5-40 nM). The association was observed for 180 seconds, and the dissociation was monitored for 300 seconds by flowing with HEPES saline buffer.

FIG. 11 illustrates the inhibitory effects of treatment with different optimized clones IL-1β-mediated downstream signaling were examined. A total of 75 pM recombinant human IL-1β was mixed with a series of diluted IgG and used to stimulate HEK-blue reporter cells for 16 hours at 37° C. following the SEAP assay. Compared with other optimization clones (IgGF4 and IgG26A), IgG26AW has the strongest inhibitory effects on IL-1β signaling.

FIG. 12 illustrates the IgG26AW binding kinetics corresponding to IL-1β from different species. Binding kinetics of IgG26AW for different species orthologs of IL-1β were estimated by Biacore 8K. IgG26AW was captured by an anti-human IgG-specific antibody immobilized on the CM5 chip. Series dilutions of different species of IL-1β were injected as the mobile phase to measure the binding of IL-1β on IgG26AW. The association was observed for 180 sec, and the dissociation was monitored for 300 sec by flowing with HEPES saline buffer.

FIG. 13 illustrates the PGG effects on IL-1β induced NF-κB signaling. A total of 75 pM recombinant human IL-1β was mixed with a series of diluted IgG (69 nM to 0 nM) and different concentration of PGG (30 μM or 50 μM) for 30 mins at 37° C. The mixture was used to stimulate HEK-blue-IL-1β reporter cells for 16 hours at 37° C. following the SEAP assay. Compared with 0 μM PGG treatment, 30 μM PGG and 50 μM PGG cotreatment with the same amount of IgG26AW shows the dose-dependent inhibitory effect on NF-κB signaling. Meanwhile, we can also observe that PGG reduce the area that cannot be completely inhibited even at high dose of IgG26AW.

FIG. 14 illustrates the PGG effects combined with low dose of IgG26AW on IL-1β induced NF-κB signaling. IL-1β was diluted from 588 to 0 pM. Low dose of IgG26AW (69 pM) combined with PGG (0, 30, 50 μM) was added with different concentration of IL-1β and incubated for 37° C. After 30 mins, the mixture was used to stimulate I-MK-blue-IL-1β reporter cells for 16 hours at 37° C. following the SEAP assay. IgG26AW (690 pM) was also used as a reference curve to demonstrate the efficient response after combined with PGG treatment.

### DETAILED DESCRIPTION OF INVENTION

The present disclosure, at least in part, is based on the development of anti-IL-1β antibodies, e.g., IgG26, IgGF4, IgG26A, IgG26AW and their variants, which possessed unexpected superior features compared with known therapeutic anti-IL-1β antibodies such as canakinumab and gevokizumab. For example, antibody IgG26AW showed higher neutralized ability to human IL-1β relative to canakinumab and gevokizumab as determined by surface plasmon resonance (SPR); potent blocking activity against the IL-1β signaling (e.g., inhibiting JNK and/or p38 phosphorylation); potent inhibitory effect of IL-1β induced cytokine production (e.g., IL-6) from immune cells (e.g., monocytes). Given the superior features of antibody IgG26AW, it would have been expected that this antibody and its functional variants would have advantageous features in blocking the IL-1β signaling and thus benefiting treatment of diseases associated with IL-1β as those described herein.

Accordingly, provided herein are antibodies capable of binding human IL-1β, as well as nucleic acid encoding such antibodies, and uses thereof for both therapeutic and diagnostic purposes. Also provided herein are kits for therapeutic and/or diagnostic use of the antibodies, as well as methods for producing anti-IL-1β antibodies.

I. Anti-IL-1β Antibodies

The present disclosure provides isolated antibodies that bind to human Interleukin-1p (IL-1β), for example, secreted IL-1β.

An antibody (interchangeably used in plural form) is an immunoglobulin molecule capable of specific binding to a target antigen (e.g., IL-1β in the present disclosure), through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multi specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The term “isolated antibody” used herein refers to an antibody substantially free from naturally associated molecules, i.e., the naturally associated molecules constituting at most 20% by dry weight of a preparation containing the antibody. Purity can be measured by any appropriate method, e.g., column chromatography, polyacrylamide gel electrophoresis, and HPLC.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinforg.uk/abs. As used herein, a CDR may refer to the CDR defined by any method known in the art. Two antibodies having the same CDR means that the two antibodies have the same amino acid sequence of that CDR as determined by the same method, for example, the IMGT definition.

In some embodiments, the isolated anti-IL-1β antibody as described herein can bind and inhibit the activity of the IL-1β by at least 50% (e.g., 60%, 70%, 80%, 90%, 95% or greater). The apparent inhibition constant (Kiapp or Ki,app), which provides a measure of inhibitor potency, is related to the concentration of inhibitor required to reduce enzyme activity and is not dependent on enzyme concentrations. The inhibitory activity of an anti-IL-1β antibody described herein can be determined by routine methods known in the art.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-IL-1β antibody described herein binds the same epitope with IL-1β antigen as a reference antibody disclosed herein (e.g., IgG26AW) or competes against the reference antibody from binding to the IL-1β antigen. An “epitope” refers to the site on a target compound that is bound by an antibody such as a Fab or full-length antibody. An epitope can be linear, which is typically 6-15 amino acid in length. Alternatively, the epitope can be conformational. An antibody that binds the same epitope as a reference antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the reference antibody. Whether two antibodies compete against each other from binding to the cognate antigen can be determined by a competition assay, which is well known in the art. Such antibodies can be identified as known to those skilled in the art, e.g., those having substantially similar structural features (e.g., complementary determining regions), and/or those identified by assays known in the art. For example, competition assays can be performed using one of the reference antibodies to determine whether a candidate antibody binds to the same epitope as the reference antibody or competes against its binding to the IL-1β antigen.

In one example, the antibody used in the methods described herein can be a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin.

Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation.

In some embodiments, the anti-IL-1β antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (e.g., human IL-1β) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (e.g., binding not detectable in a conventional assay).

In some embodiments, the antibodies described herein specifically binds to IL-1β of a specific species (e.g., human IL-1β) as relative to IL-1β from other species. For example, the antibodies described herein may specifically binds to human IL-1β as relative to mouse IL-1β. In other embodiments, the antibodies described herein may cross-react with human IL-1β and one or more IL-1β from a non-human species (e.g., a non-human primate such as macaque). In some embodiments, the antibodies cross-react with human and Rhesus macaque with similar binding affinity but have significantly lower binding affinity to mouse IL-1β. In some embodiments, an anti-IL-1β antibody as described herein has a suitable binding affinity for the target antigen (e.g., human IL-1β) or antigenic epitopes thereof.

As used herein, “binding affinity” refers to the apparent association constant or KA, which is the ratio of association and dissociation constants, K-on and K-off, respectively. The K_{A} is the reciprocal of the dissociation constant (K_{D}). The anti-IL-1β antibody described herein may have a binding affinity (K_{D}) of at least 10⁻⁸, 10⁻⁹, 10⁻¹⁰ M, 10⁻¹¹M or lower for the target antigen or antigenic epitope. For example, the anti-IL-1β antibody may have a binding affinity of 10⁻⁹M, 10⁻¹⁰ M or lower to IL-1β. An increased binding affinity corresponds to a decreased value of K_{D}. Higher affinity binding of an antibody for a first antigen relative to a second antigen can be indicated by a higher KA (or a smaller numerical value K_{D}) for binding the first antigen than the KA (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). In some embodiments, the anti-IL-1β antibodies described herein have a higher binding affinity (a higher K_{A} or smaller K_{D}) to IL-1β as compared to the binding affinity to another cytokines or chemokines (e.g., IL-6, IFN-γ, or TNFα). In some embodiments, the anti-IL-1β antibody may have a higher binding affinity to a IL-1β of a specific species (e.g., human IL-1β) than that to a IL-1β from a different species (e.g., mouse). Differences in binding affinity (e.g., for specificity or other comparisons) can be at least 1.5, 2, 2.5, 3, 4, 5, 10, 15, 20, 37.5, 50, 70, 80, 91, 100, 500, 1,000, 5,000, 10,000 or 10⁵ folds. In some embodiments, any of the anti-IL-1β antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in BBS-P buffer (10 mM HEPES pH7.4, 150 mM NaCl, 0.005% (v/v) surfactant P20) and PBS buffer (10 mM PO₄⁻³, 137 mM NaCl, and 2.7 mM KCl). These techniques can be used to measure the concentration of bound proteins as a function of target protein concentration. The concentration of bound protein ([Bound]) is generally related to the concentration of free target protein ([Free]) by the following equation:

[Bound]=[Free]/(Kd+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Provided below is an exemplary anti-IL-1β antibody IgG26AW, including its heavy chain and light chain CDR sequences and heavy chain and light chain variable domain sequences.

TABLE 1
Heavy chain and light chain CDR sequences
of exemplary anti-IL-1β antibody IgG26AW

| IgG26AW | CDR1 | CDR2 | CDR3 |
|---|---|---|---|
| Heavy chain | VDMA | WPREGWTY | NGYWNYI |
| Light chain | SWG | HTSRS | YSNFPI |

Heavy chain variable domain sequence of IgG26AW (CDRs in boldface):

EVQLVESGGGLVQPGGSLRLSCAASGFTIVDMAIHWVRQAPGKGLEWVAR
IWPREGWTYYADSVKGRFTISADTSKNTAYLQMNSLRAEDTAVYYCARFN
GYWNYIMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKAEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKDYKCKVSNKALPAPIEKTISKAKGQPREPQ
VYTLPPSRDELTRNQVSLTCLVKGFYPSDIAVEWESNGQPENNYKTTPPV
LDSDGSFFLYSKLTVDKSRWQQGNVFSCSVMHEALHNHYTQKSLSLSPGK

Light chain variable domain of IgG26AW:

DIQMTQSPSSLSASVGDRVTITCRASQDVSWGVAWYQQKPGKAPKLLIHT
SRSLYSGVPSRFSGSGSGTDFTLTISSLQPEDFATYYCQQYSNFPITFGD
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In some embodiments, an isolated anti-IL-1β antibody disclosed herein may comprise the same regions/residues responsible for antigen-binding as a reference antibody (e.g., IgG26AW), such as the same specificity-determining residues (SDRs) in the CDRs or the whole CDRs. The regions/residues that are responsible for antigen-binding can be identified from amino acid sequences of the heavy chain/light chain sequences of the reference antibody by methods known in the art. See, e.g., www.bioinf.org.uk/abs; Almagro, J. Mol. Recognit. 17:132-143 (2004); Chothia et al., J. Mol. Biol. 227:799-817 (1987), as well as others known in the art or disclosed herein. In some embodiments, the anti-IL-1β antibodies disclosed herein have the same V_{H} and/or V_{L} as a reference antibody, such as IgG26AW. In some embodiments, the anti-IL-1β antibodies disclosed herein have the same heavy chain CDRs and/or light chain CDRs as a reference antibody, such as IgG26AW.

Furthermore, the antibody may comprise specificity-determining residues that are not found in the CDR sequences of a reference antibody (e.g., IgG26AW), but are included to develop antibodies with equivalent function to the reference antibody or to further refine and optimize antibody performance. Such antibodies, as used herein, are termed SDR mutant antibodies. In general, the antibody will comprise substantially all of at least one, and typically two, variable domains, in which all or substantially all of the CDR regions and all or substantially all of the FR regions consensus sequence. The antibodies may have one or more CDRs (one, two, three, four, five, six) which are altered with respect to the original antibody. Such residues can be identified by in vitro affinity maturation of a reference antibody (e.g., IgG26AW). Methods of performing in vitro affinity maturation of a reference antibody is known in the art, see e.g., Li et al, Mabs, 2014 March-April; 6(2):437-45.

In some embodiments, the SDR mutant antibodies have at least 20%, 30%, 40%, 50%, 60%, 70%, 80%, 85%, 90%, 95%, 96%, 97%, 98%, 99%, 100%, 105% or more binging affinity to IL-1β as compared to the reference antibody, such as IgG26AW.

In some embodiments, the isolated anti-IL-1β antibody comprises a heavy chain variable region that comprises a heavy chain CDR1 (HC CDR1), a heavy chain CDR2 (HC CDR2), and a heavy chain CDR3 (HC CDR3).

In some embodiments, following the IMGT definition, the HC CDR1 may comprise the amino acid sequence of VDMA, KDNA, KDMA, DHNA, SHMA, DNAA, or NGYS.

Alternatively or in addition, the HC CDR2 may comprise the amino acid sequence of WPX₁X₂GX₃TY, in which X₁ can be Y and R, X₂ can be G and E, and X₃ can be F and W. In some examples, X₁ can be R, X₂ can be E, and X₃ can be W. Alternatively or in addition, the HC CDR3 may comprise the amino acid sequence of NGYWNYI, AGHHTGA, ALKPTSA, DSRKPRAM, GPGHTNA, or ETNPIQA.

The anti-IL-1β antibody may comprise a light chain variable region that comprises a light chain CDR1 (LC CDR1), a light chain CDR2 (LC CDR2), and a light chain CDR3 (LC CDR3). In some embodiments, following the IMGT definition, the LC CDR1 may comprise the amino acid sequence of X₄X₅G, in which X₄ can be S, A, or R and X₅ can be S, A, or R. In one example, X₄ can be S. Alternatively or in addition, the LC CDR2 may comprise the amino acid sequence of YSTAS, SQSTD, and HTSRS. In one example, the LC CDR2 can be HTSRS. Alternatively or in addition, the LC CDR3 may comprise the amino acid sequence of YSNFPI.

Also within the scope of the present disclosure are functional variants of any of the exemplary anti-IL-1β antibodies as disclosed herein. A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, anti-inflammatory activity, or a combination thereof) as the reference antibody.

In some examples, the isolated anti-IL-1β antibody disclosed herein comprises a HC CDR1, a HC CDR2, and a HC CDR3, which collectively contains no more than 10 amino acid variations (e.g., no more than 9, 8, 7, 6, 5, 4, 3, 2, or 1 amino acid variation) as compared with the HC CDR1, HC CDR2, and HC CDR3 of a reference antibody such as IgG26AW. “Collectively” means that the total number of amino acid variations in all of the three HC CDRs is within the defined range. In some examples, the anti-IL-1β antibody disclosed herein may comprise a HC CDR1, a HC CDR2, and a HC CDR3, at least one of which contains no more than 5 amino acid variations (e.g., no more than 4, 3, 2, or 1 amino acid variation) as the counterpart HC CDR of a reference antibody such as IgG26AW. In specific examples, the antibody comprises a HC CDR3, which contains no more than 5 amino acid variations (e.g., no more than 4, 3, 2, or 1 amino acid variation) as the HC CDR3 of a reference antibody such as IgG26AW.

Alternatively or in addition, the isolated anti-IL-1β antibody may comprise a LC CDR1, a LC CDR2, and a LC CDR3, which collectively contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody. In some examples, an anti-IL-1β antibody may comprise a LC CDR1, a LC CDR2, and a LC CDR3, at least one of which contains no more than 5 amino acid variations (e.g., no more than 4, 3, 2, or 1 amino acid variation) as the counterpart LC CDR of the reference antibody. In specific examples, the antibody comprises a LC CDR3, which contains no more than 5 amino acid variations (e.g., no more than 4, 3, 2, or 1 amino acid variation) as the LC CDR3 of the reference antibody.

In some embodiments, the isolated anti-IL-1β antibody disclosed herein may comprise heavy chain CDRs that collectively are at least 80% (e.g., 85%, 90%, 95%, or 98%) identical to the heavy chain CDRs of a reference antibody such as IgG26AW. Alternatively or in addition, the antibody may comprise light chain CDRs that collectively are at least 80% (e.g., 85%, 90%, 95%, or 98%) identical to the light chain CDRs of the reference antibody. In some embodiments, the anti-IL-1β antibody may comprise a heavy chain variable region that is at least 80% (e.g., 85%, 90%, 95%, or 98%) identical to the heavy chain variable region of a reference antibody such as IgG26AW and/or a light chain variable region that is at least 80% (e.g., 85%, 90%, 95%, or 98%) identical to the light chain variable region of the reference antibody.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the)(BLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the anti-IL-1β antibodies may include modifications to improve properties of the antibody, for example, stability, oxidation, isomerization and deamidation. In some instances, the antibody may comprise residues that are not found in the frame work (FR region) sequences of the reference antibody (e.g., IgG26AW).

In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York.

In some embodiments, the heavy chain of any of the anti-IL-1β antibodies as described herein may further comprise a heavy chain constant region (5 CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. In one example, the heavy chain constant region is of subclass IgG1.

The light chain of any of the anti-IL-1β antibodies described herein may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In one particular example, the anti-IL-1β antibody disclosed herein is an IgG1/kappa full-length antibody.

As described herein, the anti-IL-1β antibody can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies.

II. Preparation of Anti-IL-1β Antibodies

Antibodies capable of binding as described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., IL-1β) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the activity of IL-1β. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are XenomouseR™ from Amgen, Inc. (Fremont, Calif.) and HuMAb-Mouse™ and TC Mouse™ from Medarex, Inc. (Princeton, N.J.) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Alternatively, techniques described for the production of single chain antibodies (U.S. Pat. Nos. 4,946,778 and 4,704,692) can be adapted to produce a phage or yeast scFv library and scFv clones specific to IL-1β can be identified from the library following routine procedures. Positive clones can be subjected to further screening to identify those that inhibit IL-1β activity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the IL-1β polypeptide have been replaced (swapped) with sequences from a closely related, but antigenically distinct protein (such as CD-28 protein). By assessing binding of the antibody to the mutant IL-1β, the importance of the particular antigen fragment to antibody binding can be assessed. Alternatively, competition assays can be performed using other antibodies known to bind to the same antigen to determine whether an antibody binds to the same epitope as the other antibodies. Competition assays are well known to those of skill in the art.

In some examples, an anti-IL-1β antibody is prepared by recombinant technology as exemplified below. Nucleic acids encoding the heavy and light chain of an anti-IL-1β antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad, among others.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551(1992)] combined the tetracycline repressor (tetR) with the transcription activator (VP 16) to create a tetR-mammalian cell transcription activator fusion protein, tTa (tetR-VP 16), with the tetO bearing minimal promoter derived from the human cytomegalovirus (hCMV) promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen 5 et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-IL-1β antibody, as also described herein. The recombinant expression vector can be introduced into a suitable host cell (e.g., a dhfr-CHO cell) by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-IL-1β antibody and the other encoding the light chain of the anti-IL-1β antibody. Both of the two recombinant expression vectors can be introduced into a suitable host cell (e.g., dhfr-CHO cell) by a conventional method, e.g., calcium phosphate-mediated transfection.

Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A, Protein G or Protein L coupled matrix.

Any of the nucleic acids encoding the heavy chain, the light chain, or both of an anti-IL-1β antibody as described herein, vectors (e.g., expression vectors) containing such; and host cells comprising the vectors are within the scope of the present disclosure.

III. Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The anti-IL-1β antibody containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (produced by cellular respiration). Exemplary buffer agents include, but are not limited to a HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

In some embodiments, the buffer agent in the pharmaceutical composition described herein may maintain a pH value of about 5-8. For example, the pH of the pharmaceutical composition can be about 7.1, 7.2, 7.3, 7.4, 7.5, 7.6, 7.7, 7.8, 7.9, or 8.0. In other examples, the pharmaceutical composition may have a pH value lower than 7, for example, about 7, 6.8, 6.5, 6.3, 6, 5.8, 5.5, 5.3, or 5.

The pharmaceutical composition described herein comprises one or more suitable salts. A salt is an ionic compound that can be formed by the neutralization reaction of an acid and a base. (Skoog, D. A; West, D. M.; Holler, J. F.; Crouch, S. R. (2004). “chapters 14-16”. Fundamentals of Analytical Chemistry (8th ed.)). Salts are composed of related numbers of cations (positively charged ions) and anions (negative ions) so that the product is electrically neutral (without a net charge). An ion, as described herein, are atoms or molecules which have gained or lost one or more valence electrons giving the ion a net positive or negative charge. If the chemical species has more protons than electrons, it carries a net positive charge. If there are more electrons than protons, the species has a negative charge.

A cation (+), as described herein, is an ion with fewer electrons than protons, giving it a positive charge. (Douglas W. Haywick, (2007-2008). “Elemental Chemistry”). A cation with one positive charge can be called a monovalent cation; a cation with more than one positive charge can be called a polyvalent or multivalent cation. Non limiting examples of monovalent cations are hydrogen (H⁺), sodium (Na⁺), potassium (K⁺), ammonium (NH⁴⁺), Lithium (Li⁺), cuprous (Cu⁺), silver (Ag⁺), etc. Non limiting examples of multivalent cations are magnesium (Mg²⁺), calcium (Ca²⁺), barium (Ba²⁺), beryllium (Be²⁺), cupric (Cu²⁺), ferrous (Fe²⁺), ferric (Fe³⁺), lead(II) (Pb²⁺), lead(IV) (Pb⁴⁺), manganese(II) (Mn²⁺), strontium (Sr²⁺), tin(IV) (Sn⁴⁺), zinc (Zn²⁺), etc.

An anion, as described herein, is an ion with more electrons than protons, giving it a net negative charge. Non limiting examples of anions are azide (N³⁻), bromide (Br⁻), chloride (Cl⁻), fluoride (F⁻), hydride (H⁻), iodide (I), nitride (N⁻), Oxide (O²⁻), sulfide (S²⁻), carbonate (CO₃²⁻), hydrogen carbonate (HCO₃⁻), hydrogen sulfate (HSO₄⁻), hydroxide (OH⁻), dihydrogen phosphate (H₂PO₄⁻), sulfate (SO₄²⁻), sulfite (SO₃²⁻), silicate (SiO₃²⁻), etc.

Suitable salts for use in the pharmaceutical compositions described herein may contain a monovalent cation and a monovalent or multi-valent anion. Alternatively, the salts for use in the pharmaceutical compositions described herein may contain a monovalent or multi-valent cation and a monovalent anion. Exemplary salts include, but are not limited to, potassium chloride (KCl), sodium chloride (NaCl), calcium chloride (CaCl₂), Magnesium chloride (MgCl₂), Magnesium Sulfate (MgSO₄), Sodium Bicarbonate (NaHCO₃), Ammonium sulfate ((NH₄)₂SO₄), calcium carbonate (Ca₂CO₃), or a combination thereof.

The pharmaceutical composition described herein comprises one or more suitable surface-active agents, such as a surfactant. Surfactants are compounds that lower the surface tension (or interfacial tension) between two liquids, between a gas and a liquid, or between a liquid and a solid. Surfactants may act as detergents, wetting agents, emulsifiers, foaming agents, and dispersants. Suitable surfactants include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

A pharmaceutical composition, comprising an anti-IL-1β described herein, may comprise one or more amino acids. Exemplary amino acids include, but are not limited to, glycine, histidine, or arginine.

The pharmaceutical composition may also comprise one or more antioxidants. An antioxidant, as used herein, is an agent that prevents or delays oxidative degradation of the active ingredients contained in the composition. The antioxidants used herein may be phenolic antioxidants (sometimes called true antioxidants), reducing agents, or chelating agents. Phenolic antioxidants are sterically hindered phenols that react with free radicals, blocking the chain reaction. Reducing agents are compounds that have lower redox potentials and, thus, are more readily oxidized than the drug they are intended to protect. Reducing agents scavenge oxygen from the medium and, thus, delay or prevent drug oxidation. Chelating agents are sometimes called antioxidant synergists. Metal ions, such as Co²⁺, Cu²⁺, Fe²⁺, Fe²⁺, and Mn²⁺, shorten the induction period and increase the oxidation rate. Trace amounts of these metal ions are frequently introduced to drug products during manufacturing. Chelating agents do not possess antioxidant activity as such, but enhance the action of phenolic antioxidants by reacting with catalyzing metal ions to make them inactive.

The pharmaceutical composition described herein may also comprise a sugar derivative. A sugar derivative, as used herein, encompasses sugars and organic compounds derived from sugar. In some examples, the sugar derivative can be a non-reducing sugar, a sugar alcohol, a polyol, a disaccharide or a polysaccharide.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and mcresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN′, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules,

respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L20 glutamate, non-degradable ethylene-vinyl acetate, degradable lactic acid-glycolic acid copolymers such as the LUPRON DEPOT′ (injectable microspheres composed of lactic acid-glycolic acid copolymer and leuprolide acetate), sucrose acetate isobutyrate, and poly-D-(−)-3-hydroxybutyric acid.

In other examples, the pharmaceutical composition described herein can be formulated in a sustained release format, which affects binding selectively to tissue or tumors by implementing certain protease biology technology, for example, by peptide masking of the antibody's antigen binding site to allow selective protease cleavability by one or multiple proteases in the tumor microenvironment, such as Probody™ or Conditionally Active Biologics™. An activation may be formulated to be reversible in a normal microenvironment.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an 10 oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0. The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases.

Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

IV. Therapeutic Applications

Any of the antibodies, as well as the encoding nucleic acids or nucleic acid sets, vectors comprising such, or host cells comprising the vectors, described herein are useful for treating IL-1β mediated disorders. IL-1β mediated diseases, as used herein, refer to any medical condition associated with increased levels of IL-1β or increased sensitivity to IL-1β. Non-limiting examples of IL-1β mediated diseases are inflammatory diseases, autoimmune diseases, cancer, infectious diseases or other disorders requiring modulation of the immune response associated with IL-1β.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having inflammatory diseases, autoimmune diseases, cancer, infectious diseases or other disorders requiring modulation of the immune response. A subject having a target disease or disorder can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors for that disease/disorder.

The methods and compositions described herein may be used to treat inflammatory diseases. Non-limiting examples of inflammatory diseases are Kawasaki disease, chimeric antigen receptor T cell (CAR-T) induced cytokine release syndrome, CAR-T-induced related encephalopathy, diffuse parenchymal lung disease (DPLD), chronic obstructive pulmonary disease (COPD), aortic aneurysm, neuropathic pain, Graft-versus-host disease (GVHD), glomerulonephritis, epididymitis, atherosclerosis, erythropoietin resistance, graft versus host disease, transplant rejection, biliary cirrhosis, and alcohol-induced liver injury including alcoholic cirrhosis.

As used herein, Kawasaki disease is an illness that involves the skin, mouth, and lymph nodes, and most often affects kids under age 5. The cause is unknown, but if the symptoms are recognized early, kids with Kawasaki disease can fully recover within a few days. Untreated, it can lead to serious complications that can affect the heart.

The methods and compositions described herein may be used to treat autoimmune diseases. Examples of autoimmune diseases are cryopyrin-associated periodic syndrome, neonatal-onset multisystem inflammatory disease, rheumatoid arthritis including juvenile rheumatoid arthritis, Kawasaki disease, spondyloarthropathies including ankylosing spondylitis, inflammatory bowel diseases including ulcerative colitis and Crohn's disease, multiple sclerosis, Addison's disease, diabetes (type I), Graves' disease, Guillain-Barre syndrome, Hashimoto's disease, hemolytic anemia, systemic lupus erythematosus (SLE), lupus nephritis, myasthenia gravis, pemphigus, psoriasis, plaque psoriasis, psoriatic arthritis, autoimmune hepatitis-induced hepatic injury, rheumatic fever, sarcoidosis, scleroderma, and Sjogren's syndrome.

As used herein, “rheumatoid arthritis” refers to a type of autoimmune disease, which is characterized by synovial joint inflammations throughout the body. An early symptom of the disease is joint pain, which progresses into joint deformation, or damages in body organs such as in blood vessels, heart, lungs, skin, and muscles.

The methods and compositions described herein may be used to treat cancer. Examples of cancer are leukemia, gastric carcinoma, adenocarcinoma, mesothelioma, lung cancer, breast cancer, prostate cancer, colon cancer, head and neck cancer, melanoma, pancreatic ductal adenocarcinoma, colitis-associated colorectal cancer (CAC), or hypereosinophilic syndrome (HES). In some examples, the leukemia can be is juvenile myelomonocyte leukemia (JMML), chronic myelomonocytic leukemia (CMML) or chronic eosinophilic leukemia.

The methods and compositions described herein may be used to treat cancer. Examples of cancers that may be treated with the methods and compositions described herein include, but are not limited to: leukemia, multiple myeloma, gastric carcinoma, skin cancer, lung cancer, melanoma, renal cancer, liver cancer, myeloma, prostate cancer, breast cancer, colorectal cancer, gastric cancer, pancreatic cancer, thyroid cancer, hematological cancer, lymphoma, leukemia, skin cancer, ovarian cancer, bladder cancer, urothelial carcinoma, head and neck cancer, metastatic lesion(s) of the cancer, and all types of cancer which are diagnosed for high mutational burden. In a particular embodiment, the cancer has a high mutation burden. Subjects having or at risk for various cancers can be identified by routine medical procedures.

As used herein, “an effective amount” refers to the amount of each active agent required to confer therapeutic effect on the subject, either alone or in combination with one or more other active agents. In some embodiments, the therapeutic effect is reduced IL-1β activity, increased numbers of activated effector T cells, and/or reduced numbers or activity of regulatory T cells.

Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation.

It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the antagonist. To assess efficacy of the antagonist, an indicator of the disease/disorder can be followed.

Generally, for administration of any of the antibodies described herein, an initial candidate dosage can be about 2 mg/kg. For the purpose of the present disclosure, a typical daily, weekly, every two weeks, or every three weeks dosage might range from about any of 0.1 μg/kg to 3 μg/kg to 30 μg/kg to 100 μg/kg to 300 μg/kg to 0.6 mg/kg, 1 mg/kg, 3 mg/kg, to 10 mg/kg, to 30 mg/kg to 100 mg/kg or more, depending on the factors mentioned above. For repeated administrations over several days, weeks, months, or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 3 mg/kg every 3 weeks, followed by a maintenance dose of about 1 mg/kg of the antibody once in 6 weeks, or followed by a maintenance dose of about 1 mg/kg every 3 weeks. However, other dosage regimens may be useful, depending on the pattern of pharmacokinetic decay that the practitioner wishes to achieve. For example, dosing of 1 mg/kg once in every 3 weeks in combination treatment with at least one additional immune therapy agent is contemplated. In some embodiments, dosing ranging from about 3 μg/mg to about 3 mg/kg (such as about 3 μg/mg, about 10 μg/mg, about 30 μg/mg, about 100 μg/mg, about 300 μg/mg, about 1 mg/kg, and about 3 mg/kg) may be used. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.1 to 5.0 mg/kg may be administered. In some examples, the dosage of the anti-IL-1β antibody described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the antagonist, and the discretion of the attending physician. Typically, the clinician will administer an antibody, until a dosage is reached that achieves the desired result. In some embodiments, the desired result is a reduction of the size of the tumor, increased progression free survival period and/or overall survival. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying the development or progression of the disease or reducing disease severity.

Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In some embodiments, the antibodies described herein are administered to a subject in need of the treatment at an amount sufficient to inhibit the activity of the target antigen by at least 20% (e.g., 30%, 40%, 50%, 60%, 70%, 80%, 90% or greater) in vivo. In other embodiments, the antibody is administered in an amount effective in reducing the activity level of a target antigen by at least 20% (e.g., 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered parenterally, topically, orally, by inhalation spray, rectally, nasally, buccally, vaginally or via an implanted reservoir. The term “parenteral” as used herein includes subcutaneous, intracutaneous, intravenous, intraperitoneal, intratumor, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline. Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history. In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents. Treatment efficacy for a target disease/disorder can be assessed by methods well-known in the art.

The anti-IL-1β antibody and treatment methods involving such as described in the present disclosure may be utilized in combination with other types of therapy for the target disease or disorder disclosed herein. The term “in combination” in this context means that the antibody composition and the therapeutic agent are given either simultaneously or sequentially. Examples include chemotherapy, immune therapy (e.g. therapies involving anti-inflammatory drugs, immunosuppressant, therapeutic antibodies, antibodies, CAR T cells, or cancer vaccines), surgery, radiation, gene therapy, and so forth, or anti-infection therapy. Such therapies can be administered simultaneously or sequentially (in any order) with the treatment according to the present disclosure.

For example, the combination therapy can include the anti-IL-1β antibody and pharmaceutical composition described herein, co-formulated with and/or co-administered with, at least one additional therapeutic agent. In one embodiment, the additional agent is a cancer chemotherapeutic agent e.g. oxaliplatin, gemcitabine, docetaxel. In another embodiment, the additional agent can be disease modifying antirheumatic drugs (DMARDs) e.g. methotrexate, azathioprine, chloroquine, hydroxychloroquine, cyclosporin A, sulfasalazine, for RA treatment. Such combination therapies may advantageously utilize lower dosages of the administered therapeutic agents, thus preventing possible toxicities or complications associated with the various monotherapies. Moreover, the additional therapeutic agents disclosed herein may act on pathways in addition to or distinct from the IL-1β/NF-κB pathway, and thus are expected to enhance and/or synergize with the effects of the anti-IL-1β antibodies.

When the antibody composition described here is co-used with a second therapeutic agent, a sub-therapeutic dosage of either the composition or of the second agent, or a sub-therapeutic dosage of both, can be used in the treatment of a subject having, or at risk of developing a disease or disorder associated with the cell signaling mediated by IL-1β. A “sub-therapeutic dose” as used herein refers to a dosage, which is less than that dosage which would produce a therapeutic result in the subject if administered in the absence of the other agent or agents. Thus, the sub-therapeutic dose of an agent is one which would not produce the desired therapeutic result in the subject in the absence of the administration of the anti-IL-1β antibody described herein. Therapeutic doses of many agents that are in clinical use are well known in the field of medicine, and additional therapeutic doses can be determined by those of skill without undue experimentation. Therapeutic dosages have been extensively described in references such as Remington's Pharmaceutical Sciences, 18th ed., 1990; as well as many other medical references relied upon by the medical profession as guidance for the treatment of diseases and disorders. Additional useful agents see also Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

V. Diagnostic Applications

Any of the anti-IL-1β antibodies disclosed herein can also be used for detecting presence of IL-1β (e.g., secreted IL-1β) in vitro or in vivo. Results obtained from such detection methods can be used for diagnostic purposes (e.g., diagnosing diseases associated with secreted IL-1β) or for scientific research purposes (e.g., identifying new IL-1β secreting cell types, studying bioactivity and/or regulation of secreted IL-1β). For assay uses such as diagnostic uses, an anti-IL-1β antibody as described herein may be conjugated with a detectable label (e.g., an imaging agent such as a contrast agent) for detecting presence of IL-1β (e.g., secreted IL-1β), either in vivo or in vitro. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker.

Conjugated or attached can include covalent or noncovalent bonding as well as other forms of association, such as entrapment, e.g., of one entity on or within the other, or of either or both entities on or within a third entity, such as a micelle.

In one example, an anti-IL-1β antibody as described herein can be attached to a detectable label, which is a compound that is capable of releasing a detectable signal, either directly or indirectly, such that the aptamer can be detected, measured, and/or qualified, in vitro or in vivo. Examples of such “detectable labels” are intended to include, but are not limited to, fluorescent labels, chemiluminescent labels, colorimetric labels, enzymatic markers, radioactive isotopes, and affinity tags such as biotin. Such labels can be conjugated to the aptamer, directly or indirectly, by conventional methods.

In some embodiments, the detectable label is an agent suitable for detecting IL-1β secreting cells in vitro, which can be a radioactive molecule, a radiopharmaceutical, or an iron oxide particle. Radioactive molecules suitable for in vivo imaging include, but are not limited to, ¹²²I, ¹²³I, ¹²⁴I, ¹²⁵I, ¹³¹I, ¹⁸F, ⁷⁵Br, ⁷⁶Br, ⁷⁷Br, ²¹¹At, ²²⁵Ac, ¹⁷⁷Lu, ¹⁵³Sm, ¹⁸⁶Re, ¹⁸⁸Re, ⁶⁷Cu, ²¹³Bi, ²¹²Bi, ²¹²Pb, and ⁶⁷Ga. Exemplary radiopharmaceuticals suitable for in vivo imaging include ¹¹¹In Oxyquinoline, ¹³¹I Sodium iodide, ⁹⁹mTc Mebrofenin, and ⁹⁹mTc Red Blood Cells, ¹²³I Sodium iodide, ⁹⁹mTc Exametazime, ⁹⁹mTc Macroaggregate Albumin, ⁹⁹mTc Medronate, ⁹⁹mTc Mertiatide, ⁹⁹mTc Oxidronate, ⁹⁹mTc Pentetate, ⁹⁹mTc Pertechnetate, ⁹⁹mTc Sestamibi, ⁹⁹mTc Sulfur Colloid, ⁹⁹mTc Tetrofosmin, Thallium-201, or Xenon-133.

The reporting agent can also be a dye, e.g., a fluorophore, which is useful in detecting a disease mediated by IL-1β secreting cells in tissue samples.

To perform a diagnostic assay in vitro, an anti-IL-1β antibody can be brought in contact with a sample suspected of containing IL-1β, e.g., IL-1β secreting cells or soluble IL-1β in disease microenvironment. The antibody and the sample may be incubated under suitable conditions for a suitable period to allow for binding of the antibody to the IL-1β antigen. Such an interaction can then be detected via routine methods, e.g., ELISA, histological staining or FACS.

To perform a diagnostic assay in vivo, a suitable amount of anti-IL-1β antibodies, conjugated with a label (e.g., an imaging agent or a contrast agent), can be administered to a subject in need of the examination. Presence of the labeled antibody can be detected based on the signal released from the label by routine methods.

To perform scientific research assays, an anti-IL-1β antibody can be used to study bioactivity of IL-1β, detect the presence of IL-1β intracellularly, and or regulating the effect of secreted IL-1β. For example, a suitable amount of anti-IL-1β can be brought in contact with a sample (e.g. a new cell type that is not previously identified as IL-1β producing cells) suspected of producing IL-1β. The cells are permeabilized prior to contacting the anti-IL-1β antibody. The antibody and the sample may be incubated under suitable conditions for a suitable period to allow for binding of the antibody to the IL-1β antigen. Such an interaction can then be detected via routine methods, e.g., ELISA, histological staining or FACS.

VI. Kits for Therapeutic and Diagnostic Applications

The present disclosure also provides kits for the therapeutic or diagnostic applications as disclosed herein. Such kits can include one or more containers comprising an anti-IL-1β antibody, e.g., any of those described herein.

In some embodiments, the kit can comprise instructions for use in accordance with any of the methods described herein. The included instructions can comprise a description of administration of the anti-IL-1β antibody to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of an anti-IL-1β antibody generally include information as to dosage, dosing schedule, and route of administration for the intended treatment. The containers may be unit doses, bulk packages (e.g., multi-dose packages) or subunit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder treatable by modulating immune responses, such as autoimmune diseases. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like.

Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-IL-1β antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

Also provided herein are kits for use in detecting secreted IL-1β in a sample. Such a kit may comprise any of the anti-IL-1β antibodies described herein. In some instances, the anti-IL-1β antibody can be conjugated with a detectable label as those described herein. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

Alternatively or in addition, the kit may comprise a secondary antibody capable of binding to anti-IL-1β antibody. The kit may further comprise instructions for using the anti-IL-1β antibody for detecting secreted IL-1β.

IV. General Techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

Materials and Methods

Recombinant Human Interleukin-113 Preparation

The IL-1β cDNA (NM 000576.2, Sino Biological) was used as a template to amplify the IL-1β nucleotide codons (117-269 amino acids) without the pro-peptide region for constructing the expression plasmid. The amplified DNA was cloned into the plasmid pGEX6p-1 with BamHI and EcoRI sites, resulting in the pGEX6p-1-IL-1β vector. The proteins were expressed in BL21(DE3) cells at 16° C. for 16 hours under the induction of 1.0 mM IPTG (isopropyl β-Dthiogalactopyranoside).

For the purification of the recombinant human IL-1β, the bacterial pellet was resuspended in 20 mM Tris-HCl at pH 8.0 and lysed with a French Press. The supernatant was clarified by centrifugation (16,000 rpm, 25 min at 4° C.) and filtered by a 0.22 μm filter membrane. A GST 4 Fast Flow bead affinity column (GE Healthcare, 25 mL) was used to purify the GST-IL-1β protein in 1×PBS, pH 7.4 binding buffer, following standard manual instructions. The GST tag was removed by adding PreScission Protease (GE Healthcare, 100 μL, for 100 mg recombinant GST-IL-1β protein) and dialyzed against 5 L PBS pH 7.4 dialysis buffer for 16 hours. All dialysis samples were reloaded into a GST 4 Fast Flow affinity column twice, and the flow-through (containing active IL-1β, 17 kDa) was collected. The protein flow-through were concentrated to 8-10 mg/mL and reloaded on a Superdex75 column (GE Healthcare) to separate the remaining GST from the IL-1β. Pure IL-1β was concentrated, filtered with a 0.22 μm membrane and stored at −20° C. for cell-based assays or crystallization with IgG.

Selection and Characterization of Anti-IL-1β scFv

Several phage-display synthetic antibody libraries were screened against the immobilized recombinant human active IL-1β. The enrichment of antibody displaying phage pools specific for IL-1β was determined in round three and subsequent rounds by measuring the ratio of recovered pools of phage clones specific for IL-1β over those specific for binding BSA. Clones that bound to hIL-1β but not to BSA were subjected to DNA sequence analysis. Colonies of Escherichia coli XL1 Blue (StrateGene) harboring phagemids were inoculated directly into 150 μL of 2YT broth supplemented with carbenicillin and the M13-KO7 helper phage; the cultures were grown overnight at 37° C. in a 96-well plate. Culture supernatants containing scFv were filtered through a 0.22 μm filter and further tested for IL-1β signal neutralization in a HEK cell-based assay.

IgG Cloning, Expression and Purification

The V-regions were cloned into human IgG1κ constant regions resulting in human mAbs. The genes encoding human canakinumab (patent US20090232803 as revealed by Novartis) and gevokizumab antibody (patent WO 2008077145 as revealed by Thomson Reuters Pharma™) were synthesized. The cDNAs for the variable domains of light chain (LC) and heavy chain (HC) were amplified from the scFv plasmid of potential phages by PCR and then cloned into the mammalian expression vector pIgG (a gift from Dr. Tse-Wen Chang, Genomic Research Center of Academia Sinica). The VL domain cDNA was amplified by PCR with KOD Hot Start DNA Polymerase (Novagen) using the primers: VL-F-KpnI (5′-CAGGTGCACGATGT GATGGTACCGATATTCAAATGACCCAGAGCCCGAGCAGCCTGAGC-3′) and VL-R (5′-TGCAGCCACCGTACGTTTGATTTCCACCTTGGTGCC-3′). The VH domain cDNA was amplified by PCR using the primers: VH-F (5′-CGTGTCGCATCTGAAGTGCAGCTGGTGGAATCGGGA-3′) and VH-R-NheI (5′-GACCGATGGGCCCTTGGTGCTAGCCGAGCTCACGGTAACAAGGGTGCC-3′). PCR experiments were performed in a volume of 50 mL with 10 ng DNA template and 125 ng of each primer for 25 cycles (30 sec for 95° C., 30 sec for 55° C., 30 sec for 72° C.) followed a 10 min final synthesis step at 72° C. The PCR products were extracted from a 1.0% agarose electrophoresis gel. The linker DNA fragment between the VL and VH domains was synthesized from the pIgG vector by PCR amplification as described above by using the primers: IgG-Linker-F (5′-AAGGTGGAAATCAAACGTACGGTGGCTGCACCATCTGTC-3′) and IgGLinker-R (5′-CTGCACTTCAGATGCGACACGCGTAGCAACAGC-3′). The above three DNA fragments (VL domain, linker, and VH domain) were assembled by PCR amplification using the VL-F-KpnI and VH-R-NheI primers for 30 cycles (30 sec for 95° C., 30 sec for 56° C., 90 sec for 72° C.). The final PCR products were extracted from a 1% agarose electrophoresis gel and cloned into the pIgG vector digested by KpnI and NheI. The linearized pIgG vector and insert fragments were mixed with 4 μL Gibson Assembly Master Mix (New England BioLabs Inc., Ipswich, Mass., USA) and incubated at 50° C. for 1 hour. Half of the volume of ligation mixture was transformed with Escherichia coli JM109 competent cells. The correct clones were determined by nucleotide sequencing and were transfected into suspension HEK293 cells. Suspension HEK293 Freestyle (293F, Life Technologies, USA) cells were grown in serum-free Freestyle 293 expression media (Life Technologies) at 37° C., shaken at 110 rpm in an 8% CO₂ incubator (Thermo Scientific). For 100 mL culture transfection, suspension 293F cells in 500 mL Erlenmeyer flasks were adjusted to a density of 1.0×10⁶ cells/mL. The 100 μg plasmid DNA was diluted in 5 mL serum-free medium and filtered with a 0.2 μm syringe filter. The DNA solution was mixed vigorously with 5 mL medium containing 1 mg of cationic polymer polyethylenimine (PEI, Polysciences). After incubating for 20 min at room temperature, the DNA/PEI mixture was added dropwise to the cells with slight shaking. After 24 hours of posttransfection, tryptone N (ST Bio, Inc., Taipei, Taiwan) was added into the transfected cell culture to a final concentration of 0.5%. After 6 days of culturing, the supernatant was collected by centrifuging at 8,000×g for 30 min and filtered through a 0.45 μm membrane filter. The supernatant was loaded onto a MabSelect SuRe LX protein A affinity column (GE Healthcare) and eluted with IgG elution buffer (Pierce) into a 1/10 volume of 1 M Tris-HCl buffer at pH 9.0. The IgG proteins were further purified with a Superdex 200 gel filtration column (10/300 GL, GE Healthcare) to remove high molecular weight aggregates.

In Vitro IL-1β Neutralization Assay

HEK-blue IL-1β cells (InvivoGen) allow us to detect bioactive IL-1β by monitoring the activation of the NK-κB and AP-1 pathways. Cells were maintained in DMEM supplemented with 10% FBS, 100 μg/mL Zeocin and 200 μg/mL hygromycin B. When used for IL-1β neutralization assays, cells were seeded in 96-well plates at 3×10⁴ cells/well in 200 μL medium and incubated for 16 hours at 37° C. in a 5% CO₂ humidified incubator. Cells were then treated with 50 pM recombinant human IL-1β in the presence or absence of various concentrations of test antibodies for another 16 hours. IL-1β-induced release of secreted embryonic alkaline phosphatase (SEAP) in the supernatant was then collected and assayed by adding QUANTIBlue (InvivoGen) according to the manufacturer's protocol. Native IL-1β secreted from THP-1 cells by LPS stimulation was also provided as an IL-1β source to induce HEK-blue IL-1β cells to test the neutralization ability of different IgGs.

EC50 for Antibody-Antigen Interactions

The IgG EC50 was determined by titrating IgG antibodies on immobilized 500 ng IL-1β with ELISA. Briefly, IL-1β (500 ng/well) was coated with PBS buffer (pH 7.4) on NUNC 96-well Maxisorp immuno plates overnight at 4° C. and blocked with 5% skim milk in PBST [0.05% (v/v) Tween 20] for at least 1 hour. Simultaneously, IgG in PBST with 5% skim milk was prepared at 11 concentrations by two-fold serial dilutions. After blocking, 100 μL diluted IgG samples were added to each well coated with IL-1β and incubated for another 1 hour under gentle shaking. The plates were washed 6 times with 300 μL PBST and then 100 μL 1:5000-diluted horseradish peroxidase/anti-human IgG antibody conjugated in PBST with 5% milk was added and incubated for 30 mins. The plates were washed six times with PBST buffer and twice with PBS, developed for 3 min with 3,3′,5,5′-tetramethyl-benzidine peroxidase substrate (Kirkegaard & Perry Laboratories), quenched with 1.0 M HCl and read spectrophotometrically at 450 nm. The EC50 (ng/mL) was calculated according to the Stewart and Watson method.

Binding Affinity Determination by Surface Plasmon Resonance (SPR)

IL-1β binding experiments of different IgGs were performed on a Biacore T100 instrument (GE Healthcare). Recombinant IL-1β was produced from our lab and diluted in running buffer to six concentrations (2.5-40 nM). A biosensor surface was prepared by immobilizing an antihuman IgG specific antibody on flow cells 3 and 4 of a CM5 chip by using the human antibody capture kit (BR100839, GE Healthcare) and amine coupling kit (BR100050, GE Healthcare) according to the manufacturer's instructions. All IgG samples were dialyzed against 10 mM HEPES, pH 7.4, 150 mM NaCl, 3 mM EDTA, 0.005% Surfactant P20 and 0.02% BSA before measurements. All experiments were carried out with a flow rate of 10 μL/min at 25° C. The association of IL-1β on both flow cells was monitored for 180 sec, and the dissociation was monitored for 300 sec by subsequently flowing with HEPES saline buffer. The sensorgrams were double-referenced against the reference flow cell and fit globally to a 1:1 binding model to generate kinetic rate constants by using the Biacore T100 evaluation software version 1.0 (Biacore).

Crystallization and Data Collection

To obtain the Fab/IL-1β complex crystal structures, Fab domains of the potential antibodies, 26 and 26A, were prepared for crystallization. Purified Fab and IL-1β were premixed at a 1:1 molar ratio at 4° C. overnight. The mixture was loaded onto the gel-filtration column (Superdex 200 prep-grade XK16/70, GE Healthcare), and the protein complex was eluted at a flow rate of 0.3 mL/min at 4° C. in a buffer solution consisting of 50 mM Tris and 100 mM NaCl at pH 8.0. The optical absorbance at 280 nm was used to monitor the eluted protein complex.

The Fab/IL-1β complex crystals were grown by mixing 1 μL protein solution with 1 μL reservoir solution using the sitting-drop vapor-diffusion method at 293 K. Crystals of the 26-Fab/IL-1β complex were obtained in a reservoir solution consisting of 17% (w/v) PEG 3350, 10% (v/v) glycerol, and 0.1 M citric acid at pH 3.8. Crystals of the 26A-Fab/IL-1β complex were obtained in a reservoir solution consisting of 17% (w/v) PEG 3350, 10% (v/v) glycerol, and 0.1 M citric acid at pH 4.0. All crystals were flash-cooled, and the diffraction patterns were recorded at cryogenic temperatures. The diffraction data of 26-Fab/IL-1β crystals were collected at a wavelength of 1.0 Å on the Taiwan Photon Source (TPS) beamline TPS-05A at the National Synchrotron Radiation Research Center (NSRRC) in Taiwan using a Rayonix MX300-HS CCD detector. The diffraction data of 26A-Fab/IL-1β crystals were collected at a wavelength of 0.9 Å on the beamline BL44XU of the SPring-8 synchrotron in Japan using an MX-225 CCD detector. Diffraction data were processed and scaled using HKL-2000.

Structure Determination and Refinement

The 26-Fab/IL-1β complex crystal structure was determined by molecular replacement (MR) using the software MOLREP in the CCP4 suite₂₄ with the IgG1-Fab (PDB ID: 2FJF) and the human IL-1β (PDB ID: 2I1B) fragments as the search models. The 26-Fab/IL-1β complex crystals belonged to C2 space group with one 26-Fab/IL-1β complex in an asymmetric unit. Throughout the refinement using REFMACS in the CCP4 suite, a randomly selected 5% of the data were set aside for cross-validation by the R_{free} value. Manual modifications of the models were performed using the program Coot. The complex structure was refined to a resolution of 2.65 Å, from which R_{work} and R_{free} values of 22.9 and 27.5%, respectively, were obtained. The 26A-Fab/IL-1β crystal structure was determined by the MR method using the refined 26-Fab/IL-1β complex structure as the search model. The 26A-Fab/IL-1β complex structure was refined to a resolution of 2.48 Å with R_{work} and R_{free} values of 21.2 and 27.6%, respectively. Data collection and final model statistics are shown in Table 2. The molecular figures were produced using Chimera.

Cytokine Biomarker Assay

Male C57BL/6 mice (Jackson Laboratory, Bar Harbor, Me.) were pretreated intravenously with the neutralizing antibody (canakinumab and IgG26AW) and control IgG (isotype IgG) at 0 and 0.2 mg/kg simultaneously (n=9 mice in the pretreatment group). Then, the mice were injected intraperitoneally with recombinant human IL-1β (R&D systems) 240 ng/200 μL/mouse; then, 2.5 hours after the injection (peak IL-6 response time), the mice were restrained, and blood was collected via a facial vein with a lancet. Serum mouse IL-6 levels were measured using a Quantikine ELISA Kit (R&D System) according to the manufacturer's protocol.

Analysis of Phosphoproteins after IL-1β Stimulation in A549 Cells

A549 cells were obtained from the American Type Culture Collection (ATCC) and grown in 10% DMEM (Gibco) with 10% fetal bovine serum. Phosphorylation of p38, IRAK4 and JNK kinase and total IκB degradation were assessed by Western blotting (n=3). Antibodies against tubulin, phospho-p38 (p-p38), phospho-JNK, p38 and JNK used in Western blotting were purchased from Cell Signaling Technology; the anti-human IκB-α antibody was obtained from R&D Systems. An equal amount of protein in each sample (30 μg of whole cell lysate/well) was separated by 12% SDS-PAGE and transferred to a PVDF membrane. Membranes were blocked in Tris-buffered saline buffer containing 0.1% Tween 20 and 5% bovine serum albumin and probed with anti-p-p38 and anti-p-JNK. Other antibodies were diluted in Tris-buffered saline buffer containing 0.1% Tween 20 and 5% low fat milk and probed on PVDF membranes. Following the incubation with a peroxidase-conjugated secondary antibody, proteins were visualized with a chemiluminescence detection system.

Lung (Xenograft) and Breast Cancer (Orthotopic) Models in Nude and ASID Mice

Human A549 tumor cells and human MDA-MB-231 cells were acquired from American Type Culture Collection (ATCC) and used to establish the xenograft model. Both of the cell types were grown in DMEM medium (Gibco) containing 10% fetal bovine serum (Gibco) at 37° C. in a humidified incubator containing 5% CO₂. A total of 5×10⁶/100 μL A549 cells were inoculated into the subcutis of 6-8-week-old male nude mice. A total of 1×10⁶/100 μL MDAMB-231 cells were orthotopically injected into the mammary fat pad in ASID mice. ASID mice were produced by breeding NOD.CB17-PrkdcSCID/JNarl with B6.12954-Il2rgtm1Wjl/J that carried the X-linked Il2rg mutation and maintained by the National Applied Research Laboratories (Academia Sinica). B cells, T cells, and NK cells are deficient in ASID mice, and they are superior to traditional immunodeficient mice for human tumor transplantation. When the tumor reached a suitable tumor size of 80-90 mm³, the mice were randomly assigned into control and treatment groups. A549 and MDA-MB-231 tumor-bearing mice were treated three times a week for a total of 5 weeks for dosages with isotype IgG and IgG26AW antibodies (10 mg/kg). The diameter of the tumors and weight changes were measured twice per week, and tumor volume (V) was calculated using the following formula: V=ab²/2, where a is the longest diameter of the tumor, and b is the shortest diameter of the tumor. Tumor masses greater than 3 mm in diameter were recorded. All mouse experiments were conducted according to relevant guidelines and experimental protocols approved by the Institutional Animal Care and Utilization Committee (IACUC) of Academia Sinica (Protocol IDs: NLAC(TN)-106-D-011 and 106-NLAC-EN-060).

Statistical Analysis

Statistical analyses and graphical representation of data were carried out using GraphPad Prism version 6.0 (GraphPad Software). Data are presented as the mean±standard deviation (SD) of at least three independent experiments. Statistical significance was calculated using a multiple comparison t-test. P-values less than 0.05 were considered statistically significant.

### Example 1: Selection of Human Generic Phage Libraries Identifies IgG26 with the Inhibitory Potential on the IL-1β Signaling Pathway

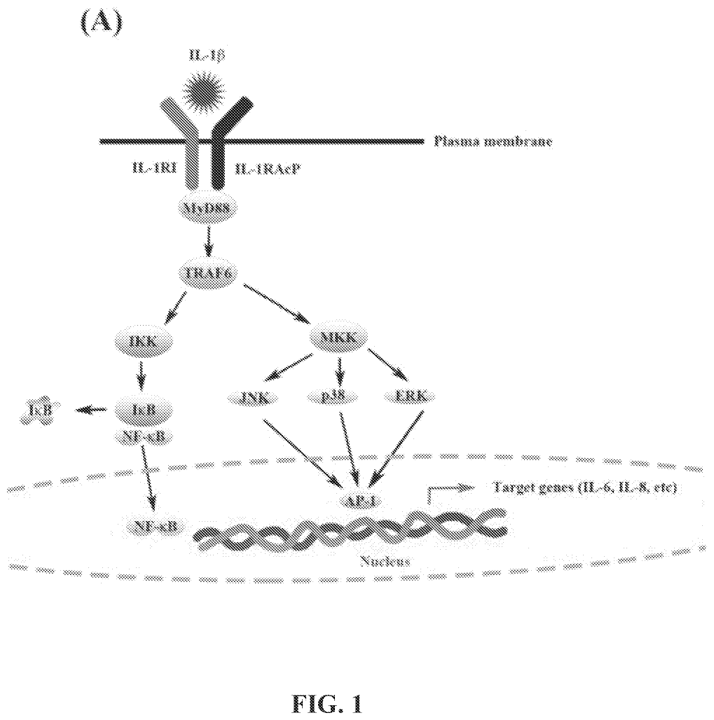

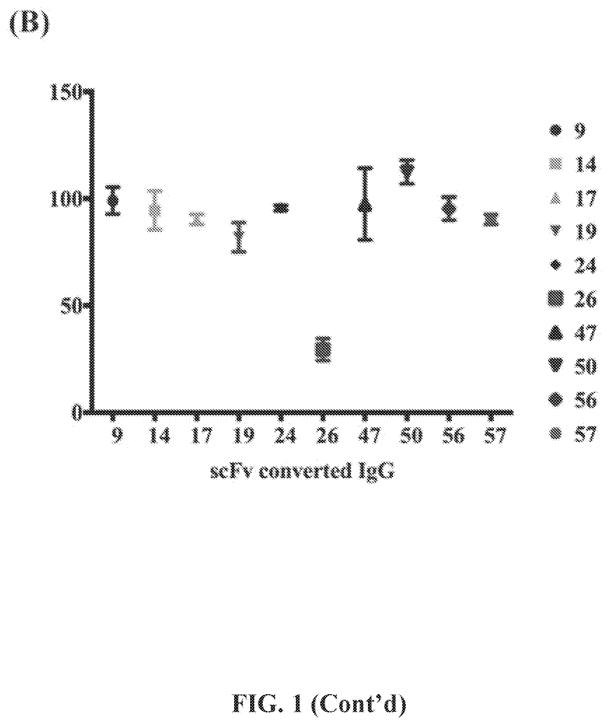

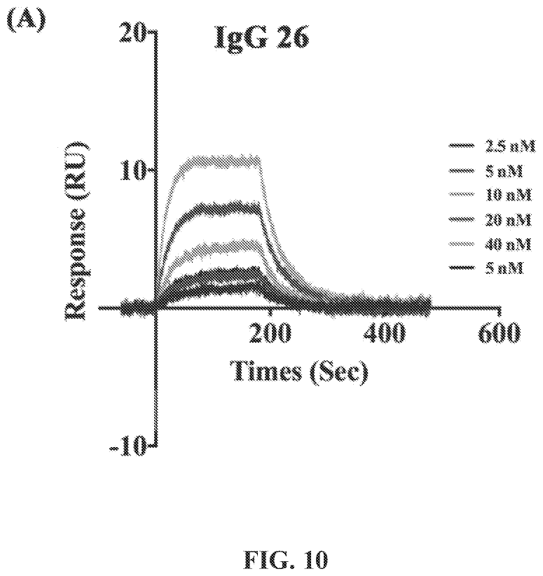

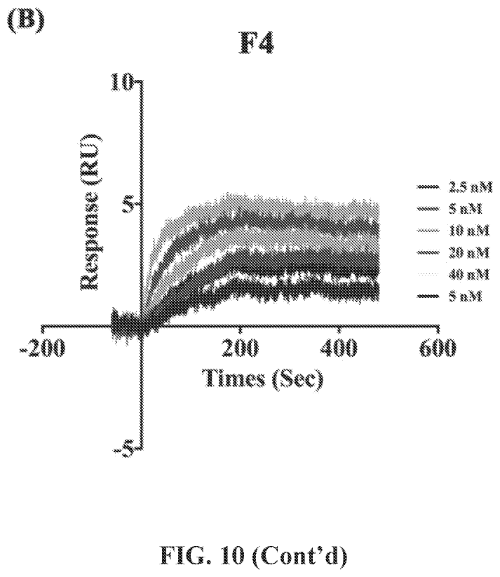

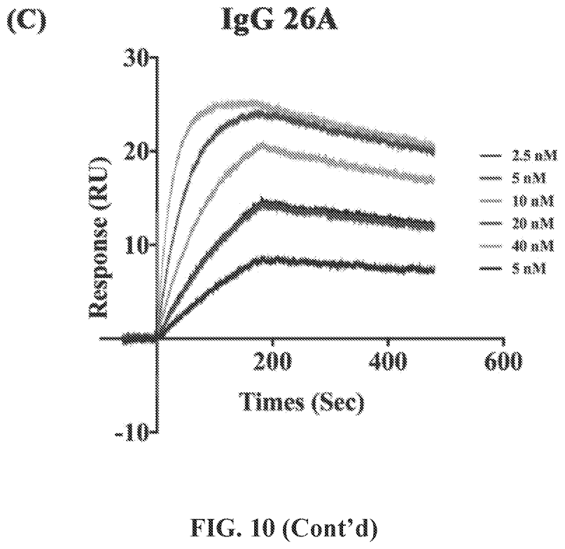

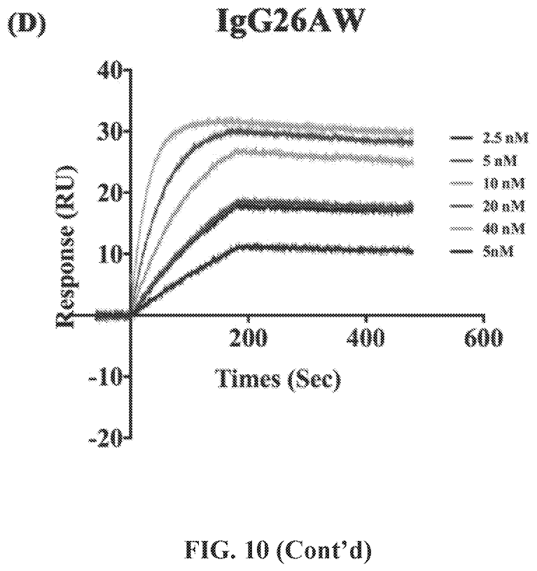

The functional scFv IL-1β-binders were selected from the GH2 artificial phage-displayed human antibody libraries established from the idea of bearing the characteristics of the natural antibody repertoire. The GH2 synthetic antibody libraries were designed based on computational analyses and experimental investigations and were constructed for discovering highly functional antibodies targeting many antigens on diverse epitopes. After 3 rounds of selections against recombinant human active IL-1β protein, we isolated 10 scFvs able to bind to human IL-1β. After reformatting into human IgG1 antibodies with the IGKV-1-NL*01/IGHV3-23*04 framework for the V_{L} and V_{H} variable domains, not only do scFvs bind IL-1β but we also hope that the binding site of scFvs can interfere with IL-1β-induced downstream signaling. Therefore, a functional cell-based assay was performed to further screen the potential candidates. Only one of them, IgG26, had the ability to neutralize IL-1β-induced NF-κB signaling at a high dose (69 nM) (FIG. 1, part B). The binding affinity of IgG26 against recombinant human IL-1β was also monitored by SPR and showed K_{D}=10⁻⁸ M (Table 1 and FIG. 10). Therapeutic antibodies are extensively engineered to possess desirable biological and physicochemical properties, such as low immunogenicity, high affinity and specificity, optimal effector functionality, and good solubility and stability. Therefore, to better understand the inhibitory mechanism of IgG26 and to increase its strength and inhibitory ability for IL-1β binding, the antibody binding epitope on IL-1β should be identified.

TABLE 1
Comparative binding affinities and kinetics of different optimized IgGs binding to IL-10 by SPR.

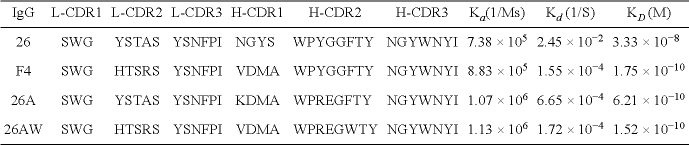

### Example 2: IgG26 Epitope Mapping by X-Ray Crystallography

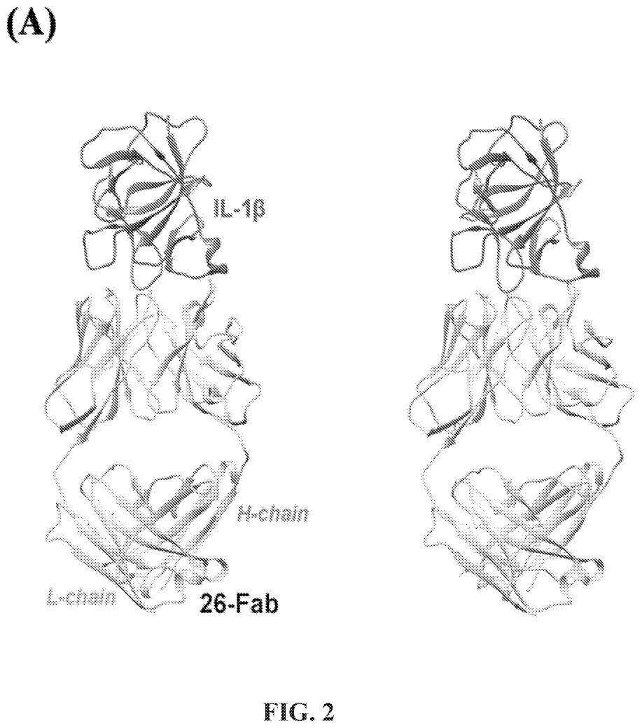

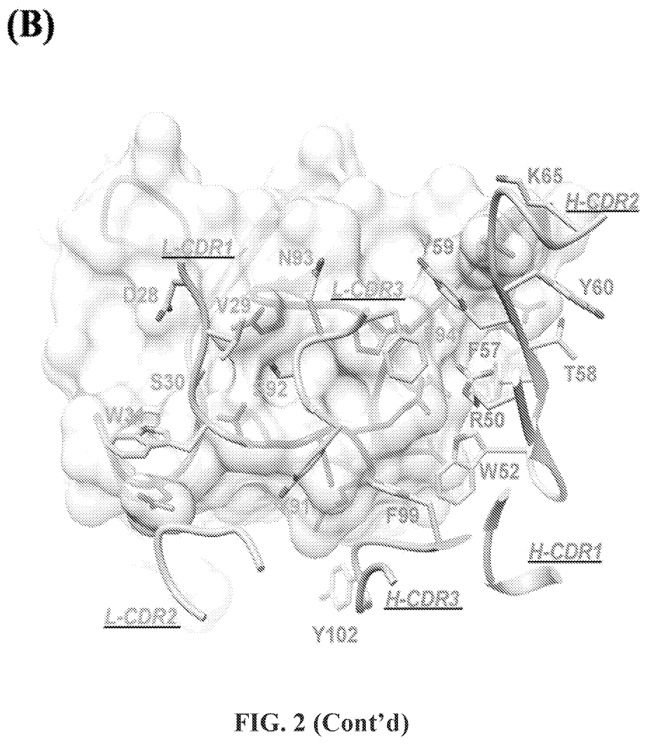

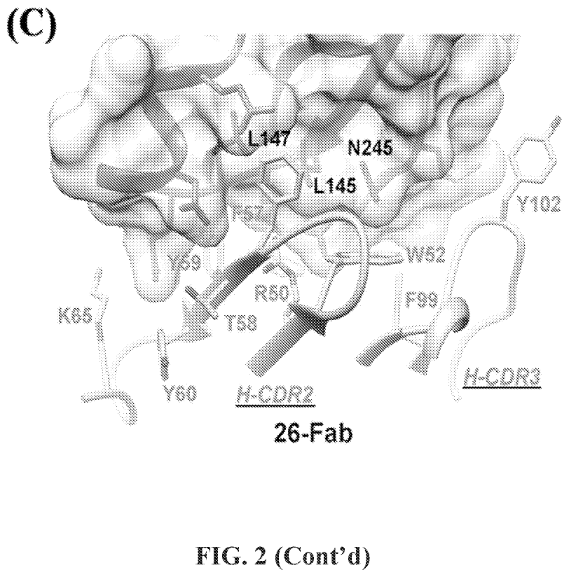

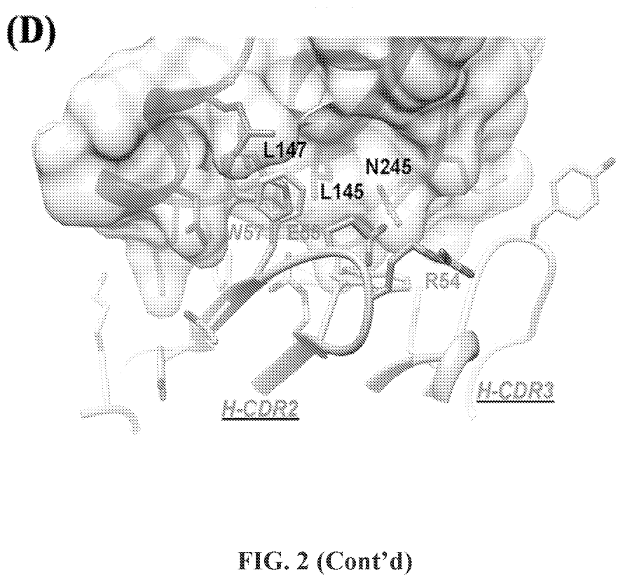

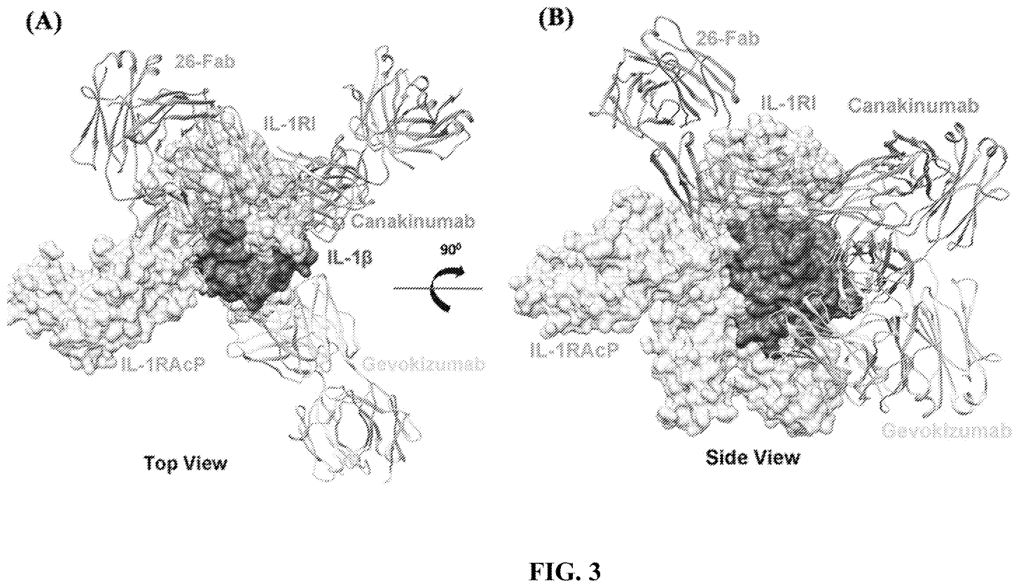

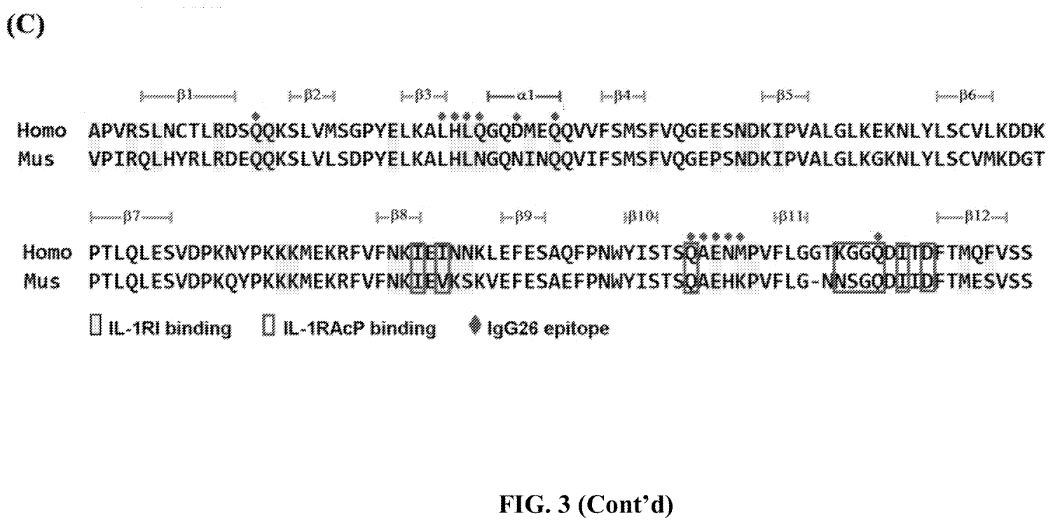

To understand how IgG26 specifically recognizes IL-1β and inhibits the IL-1β receptor signaling pathway, the Fab fragment of IgG26 (26-Fab) and an N-terminally truncated IL-1β (residues 119-268) were prepared to form the complex for crystallization. The 26-Fab/IL-1β complex was crystallized in the C2 space group and determined to have a resolution of 2.65 Å (Table 2) with one complex structure in the asymmetric unit. As shown in FIG. 2, part A, the current structure of the IL-1β molecule in the complex structure adopted a β-trefoil structure consisting of 12 β-strands with one α-helix between β3 and β4; the N-terminus starts at residue 119 and the C-terminus ends at residue 268 (FIG. 3, part C). The overall structure of IL-1β presented here is similar to that of receptor-bound IL-1β (PDB ID: 4DEP), with an R.M.S.D. value of 0.657 Å for 141 Ca atoms when the two structures were superimposed. The 26-Fab displays the typical immunoglobulin fold; the light chain CDR loops are composed of residues 30-32 (LCDR1), 49-53 (L-CDR2) and 91-96 (L-CDR3), and the CDR loops of the heavy chain are composed of residues 30-33 (H-CDR1), 52-59 (H-CDR2) and 99-106 (H-CDR3) (FIG. 3, part B). The 26-Fab binding site displays a conformational epitope located on three external regions, β1-β2 loop, β3-β4 region with an α-helix, β10-β11 loop and β11-β12 loop of IL-1β (FIG. 3, part C); an area of 2206 Å2 on IL-1β is buried by 26-Fab upon complex formation. FIG. 2, part B, shows the interaction interface of IL-1β and 26-Fab. The paratope on IgG26 consists of two light chain CDRs (L-CDR1 and L-CDR3) and two heavy chain CDRs (H-CDR2 and H-CDR3) contributing to the IL-1β-specific interaction, and L-CDR2 and H-CDR1 do not have any interactions with IL-1β. The side chains of residues S30 and W31 in the light-chain L-CDR1 make hydrogen bonds to the side chain of Q242 and the backbone 0 atom of G256 of IL-1β respectively. The side chain of IL-1β Q130 hydrogen bonds to the side chains of L-CDR1 S30 and L-CDR3 N93. The hydrophobic side chains of Y91 in L-CDR3 make nonpolar interactions with A243. In addition, the L-CDR3 F94 side chain hydrophobically interacts with E244 and stacks with the H146 side chain of IL-1β. In the heavy-chain H-CDR2, W52 also provides a strong hydrogen bond to the side chain of E244 on IL-1β. The side chain of F57 inserts into a hydrophobic cavity on IL-1β and makes strong hydrophobic interactions with residues L145 and L147. In addition, three residues, F99, G101 and Y102, on H-CDR3 provide a hydrophobic surface for the interaction of residues A243, M246 and P247 of IL-1β.

TABLE 2
Data collection and refinement statistics.

| | 26-Fab/IL-1β | | 26A-Fab/IL-1β | |
|---|---|---|---|---|
| Data collection | | | | |
| Wavelength (Å) | 1.0 | | 0.9 | |
| Space group | C2 | | C2 | |
| Cell dimensions (Å°) | a = 156.34, b = 112.24, c = 38.58, | | a = 155.69, b = 112.70, c = 38.94, | |
| | α = 90.0, β = 94.85, γ = 90.0 | | α = 90.0, β = 94.53, γ = 90.0 | |
| Resolution (Å) | 20-2.65 | (2.74-2.65) | 25-2.48 | (2.57-2.48) |
| Unique reflections | 19,167 | | 23,536 | |
| R_{merge} (%) | 5.7 | (54.7) | 5.2 | (42.1) |
| I/σ(I) | 20.7 | (2.3) | 28.8 | (4.56) |
| Completeness | 99.6 | (100) | 98.9 | (96.1) |
| Redundancy | 2.7 | (2.8) | 3.7 | (3.7) |

| CC1/2^{a} | 0.76 | 0.88 |
|---|---|---|
| CC*^{a} | 0.93 | 0.97 |
| Refinement | | |
| Resolution (Å) | 20-2.65 | 25-2.48 |
| No. of reflections R_{work}/R_{free} | 17,140/963 | 22,314/1,166 |
| R_{work}/R_{free} | 20.0/25.4 | 19.3/25.9 |
| No. of atoms/Avg B factor (Å²) | | |
| Protein | 4,428/70.8 | 4,465/57.1 |
| Water | 280/43.7 | 329/39.2 |
| RMSD | | |
| Bond lengths (Å)/Bond angles (°) | 0.004/1.26 | 0.009/1.58 |
| Ramachandran statistics (%)^{b} | | |
| Favored | 95.04 | 94.21 |
| Outliers | 2.62 | 3.98 |
| Clash score | 3.2 | 4.0 |
| MolProbity score | 1.77 | 2.04 |

^{a}Values corresponding to the highest resolution shells are shown in parentheses.
^{b}Stereochemistry of the model was validated with MolProbity.

### Example 3: IgG26 Maturation by Screening Phage-Display Optimized Libraries

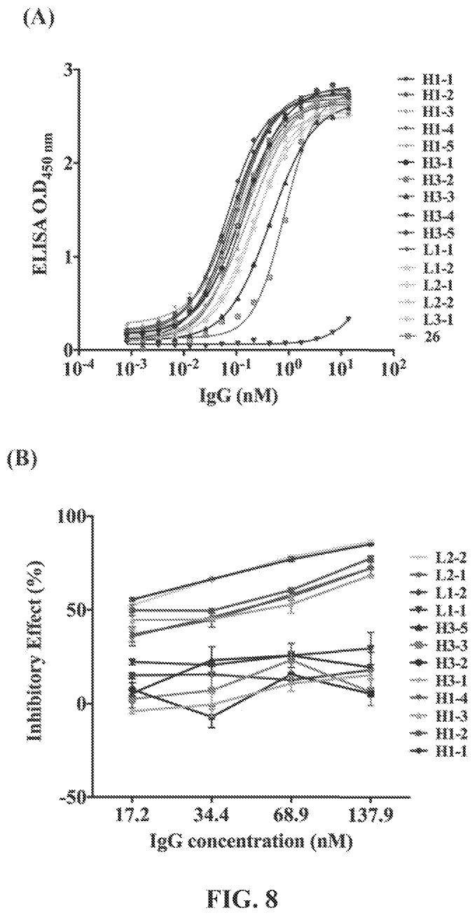

To improve the neutralization potency of IgG26 targeting human IL-1β, according to the sequence of IgG26, optimization libraries in which most of the six complementary-determining regions (CDRs) were allowed to vary in only one amino acid residue at a time were constructed to screen for mutations that improve both the binding affinity and neutralization potency. Fifteen scFvs were selected by screening three times against human IL-1β. Five of them are variants of H-CDR1, and the other five are variants of H-CDR3. For the L-CDR1, L-CDR2, LCDR3 variants, there are two, two, and one of scFvs (Table 3). Therefore, no better variants were found in the H-CDR2 region, which also corresponds to the structural analysis results. HCDR2 is too critical for IL-1β binding to be replaced by other H-CDR2 sequence combinations. We used HEK-blue IL-1β reporter cells to test the inhibitory effect of downstream signaling stimulated by IL-1β in variant antibodies. We found that sequence-optimized changes in HCDR1 and L-CDR2 were positively correlated with the ability to inhibit the IL-1β-induced downstream response (FIG. 8, part B). Therefore, we selected the best inhibitory variants of HCDR1 (H1-1) and L-CDR2 (L2-2) to combine together as IgGF4 to achieve a better binding affinity (K_{D}=1.75×10⁻¹⁰) and inhibitory ability (IC₅₀=2.72 nM) (Table 1 and FIG. 10).

TABLE 3
Phage-display CDR optimized scFvs sequence.

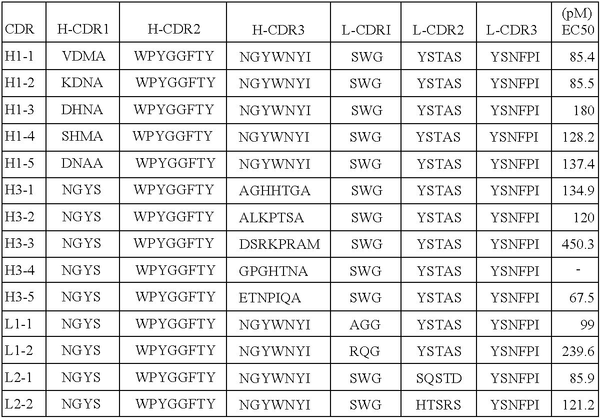

### Example 4: Structure-Based Sequence Optimization of H-CDR2

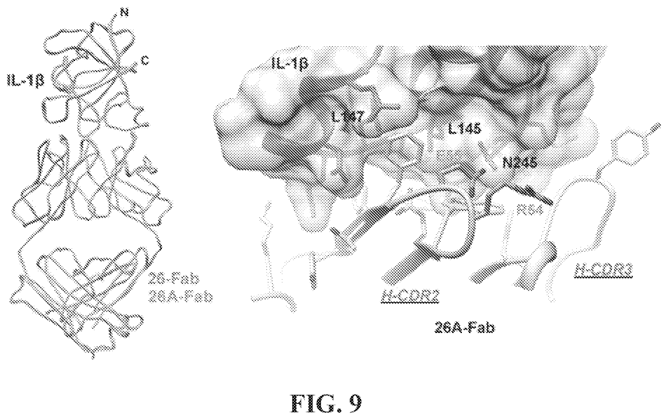

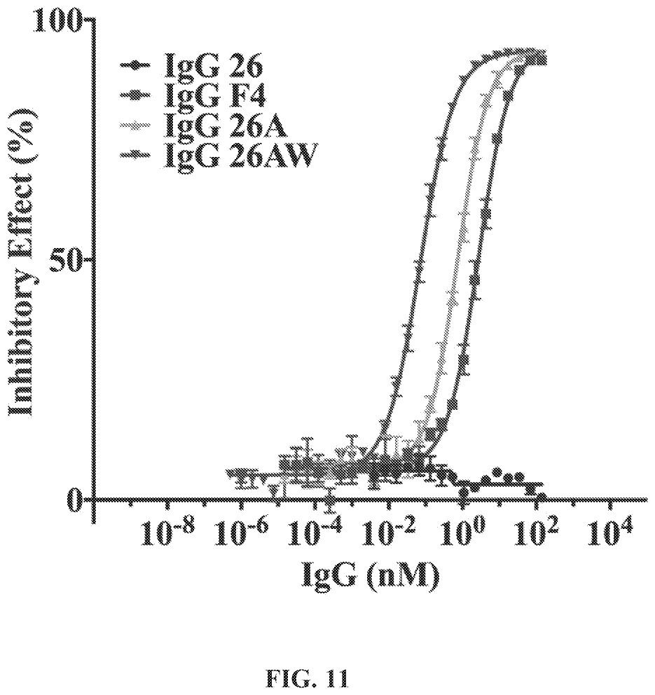

Based on the crystal structure of the 26-Fab/IL-1β complex, we found that H-CDR2 (amino acid sequence WPYGGFTY) is an important region responsible for binding to IL-1β. In this structure, W52 and F57 provide specific interactions with IL-1β (FIG. 2, part C), residues 53-56 between two large aromatic residues show no interactions with the antigen, and a cavity was observed at this region. Therefore, this H-CDR2 loop was chosen for improving the affinity by site-directed mutagenesis. P53 and G56 play a significant role in H-CDR2 to maintain the loop conformation. Therefore, a double mutation, Y54R and G55E in H-CDR2, termed IgG26A, was constructed and purified for further analysis. The 26A-Fab/IL-1β complex was also crystallized in the C2 space group under similar crystallization conditions as the 26-Fab/IL-1β complex structure. The 26A-Fab/IL-1β complex structure was determined at a 2.48 Å resolution with one Ag-Fab complex in the asymmetric unit. In the current structure, 26A-Fab binds to the identical epitope as the 26-Fab binding site on IL-1β. Here, the H-CDR2 Y54R mutation does not provide additional interactions with the antigen but may increase the protein solubility. Interestingly, the G55E mutation causes the E55 side chain to form a strong hydrogen bond to the N245 side chain of IL-1β (FIG. 9, part B). The affinity analysis (Table 1 and FIG. 10, part C) and cell-based functional assay (FIG. 11) also demonstrated that IgG26A had an association constant of 1.07×10⁶ (1/Ms) and had enhanced inhibitory activity against IL-1β signaling (IC50=0.74 nM) (Table 1 and FIG. 10).

In addition, we further created an F57W mutation in IgG26A, termed IgG26AW. The larger aromatic side chain of W57 inserted into the hydrophobic cavity may increase the strong hydrophobic interaction with residues L145 and L147 on IL-1β (FIG. 2, part D). As we suspected, the affinity analysis and cell-based functional assay suggested that IgG26AW had an equilibrium constant of 1.52×10⁻¹⁰ M and had enhanced inhibitory activity against IL-1β signaling (IC₅₀=0.071 nM) (Table 1 and FIG. 11). Finally, we obtained IgG26AW as the final version and further tested its efficacy in in vitro and in vivo systems.

### Example 5: IL-1β Signaling Inhibition Mechanism

IL-1β bound to its primary receptor IL-1RI and its receptor accessory protein IL-1RAcP to form the IL-1β-receptor signaling complex to initiate signaling. The crystal structure of the IL-1β/IL-1RI/IL-1RAcP ternary complex demonstrates the overall complex architecture and indicates important IL-1β residues that contribute to binding with two receptor molecules (PDB ID: 4DEP). Here, the 26-Fab/IL-1β complex structure was superimposed on the IL-1β/IL-1RI/IL-1RAcP ternary complex to compare the spatial overlap of Fab and receptors (FIGS. 3A and 3B). The structure of 26-Fab shows a large overlap region with IL-RI and extends to a small overlap region with IL-1RAcP. Their spatial overlap indicates the possible competition mode between IgG26 and two complex components (IL-1RI and IL-1AcP). As shown in FIG. 3, part C, several IgG26 binding residues, including Q130, H146, L147, Q148, Q154, E244, and M246, in IL-1β are also involved in its receptor IL-RI binding. Moreover, two residues, Q242 and Q257, in the epitope contributed to the accessory receptor IL-1RacP binding (PDB ID: 4DEP). IgG26 has overlapping binding areas and residues with two complex components on IL-1β. This result indicated that IgG26 bound with IL-1β blocks both IL-RI/IL-1β and IL1RAcP/IL-1β interactions to prevent the assembly of the IL-1β/IL-1RI/IL-1RAcP ternary complex.

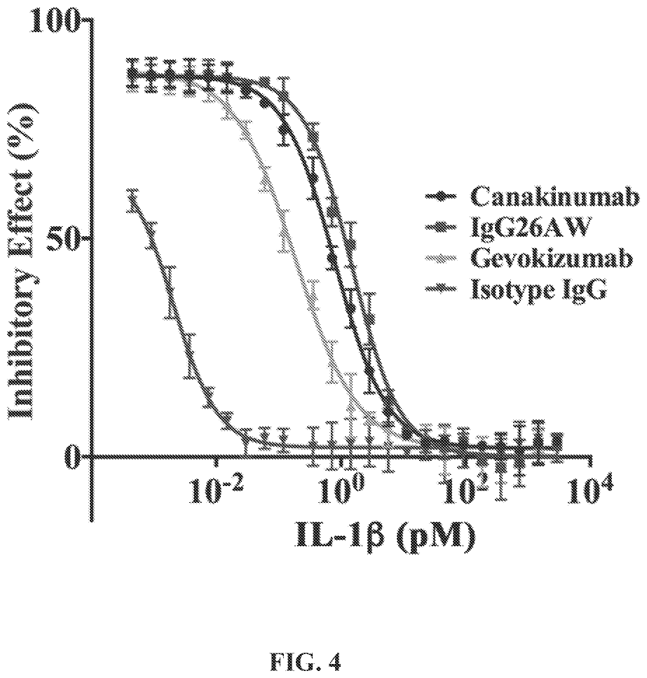

After optimization of IgG26 to IgG26AW, we further compared IgG26AW with canakinumab and gevokizumab, which were developed by NOVATIS and XOMA corporations, respectively. HEK-blue IL-1β cells were used to respond to different concentrations of IL-1β and then treated with 5 nM of IgG26AW, canakinumab, gevokizumab and isotype IgG. After 16 hours, the inhibitory effect of IL-1β-induced signaling was monitored by SEAP assay. FIG. 4 shows that IgG26AW inhibits half of the NF-κB signal produced by 1.177 nM IL-1β compared together, gevokizumab and canakinumab only inhibit half of the NF-κB signal produced by 0.3526 nM and 0.9253 nM IL-1β respectively. Accordingly, IgG26AW has a better neutralization ability of IL-1β than those of the other two candidate IL-1β inhibitors.

### Example 6: IgG26AW Activity In Vivo

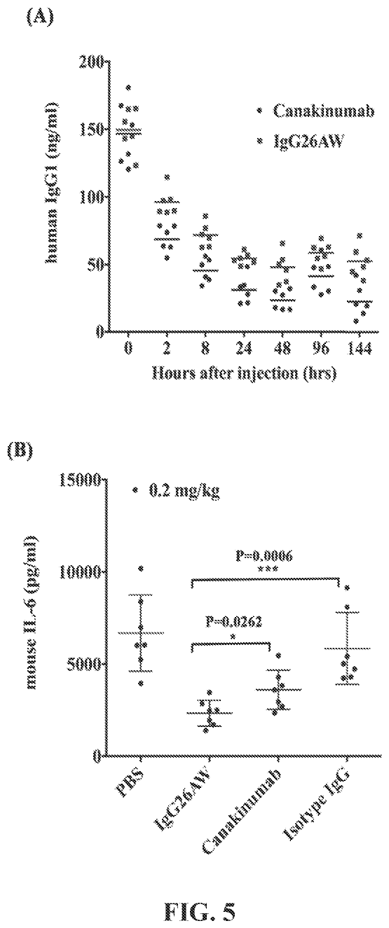

To determine whether various rodent and primate disease models could be used to test the in vivo efficacy of IgG26AW, the ability of IgG26AW to bind a number of species orthologs of IL-1β was measured by SPR. Unfortunately, IgG26AW binds to mouse IL-1β with low affinity, which was determined to be 4.51 nM (Table 4). This affinity is not sufficient to test IgG26AW efficacy in mouse disease models. Therefore, to assess whether IgG26AW could systemically neutralize human IL-1β an in vivo C57BL/6 mouse cytokine biomarker assay was performed due to the mouse IL-1 receptor cross-reacting with human IL-1β. The mice were pretreated with IgG26AW, canakinumab, or isotype control IgG by intravenous injection and further challenged with an exogenous dose of human IL-1β; then, the induction of murine IL-6 was measured in serum. FIG. 5, part B, shows that IgG26AW blocked the increase in human IL-1β-induced systemic IL-6 expression in the mice, with 65% inhibition at 0.2 mg/kg injected antibody, to a greater extent than canakinumab (47% inhibition) (FIG. 5, part B). Moreover, concentrations of IgGs after the injection in the serum were evaluated in different time courses. Both IgG26AW and canakinumab showed similar serum concentrations and IgG stability at different time courses in the mouse sera (FIG. 5, part A). This indirect evidence suggested that IgG26AW has significant efficacy in a murine disease model in which IL-1β has a critical role in the induction and maintenance of pathology. Additionally, compared with the commercial IgG canakinumab, IgG26AW more strongly blocked IL-1β-induced IL-6 secretion in mice at the same drug concentration, indicating that IgG26AW has the potential to be further developed into a therapeutic drug.

TABLE 4
The IgG26AW binding kinetics against to different species IL-1β

| Species | Kₐ (1/Ms) | K_{d} (1/s) | K_{D} (M) |
|---|---|---|---|
| Human IL-1β | 7.38 × 10⁶ | 2.54 × 10⁻⁴ | 2.5 × 10⁻¹⁰ |
| Mouse IL-1β | 8.23 × 10² | 3.72 × 10⁻³ | 4.51 × 10⁻⁶ |
| Rabbit IL-1β | 7.40 × 10⁴ | 4.91 × 10⁻³ | 6.63 × 10⁻⁸ |
| Monkey IL-1β | 4.37 × 10⁵ | 2.76 × 10⁻⁴ | 6.31 × 10⁻¹⁰ |
| Canine IL-1β | 4.11 × 10⁵ | 1.73 × 10⁻² | 4.21 × 10⁻⁸ |

### Example 7: IgG26AW Inhibits Human Lung Cancer Progression

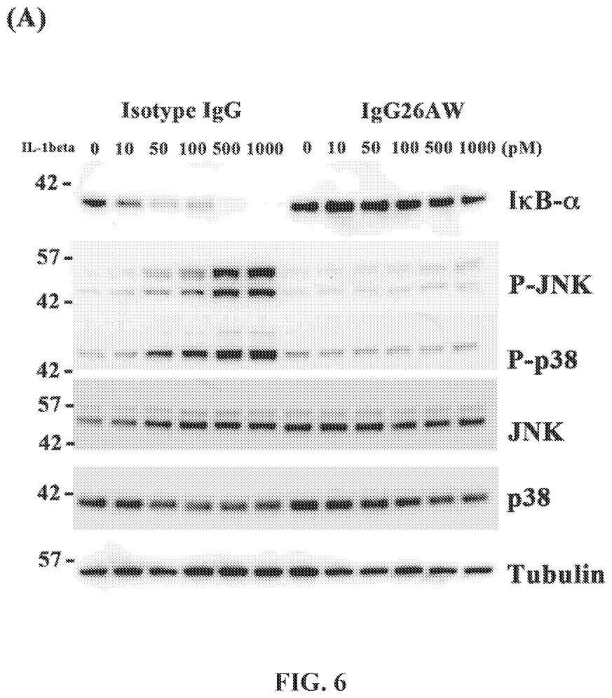

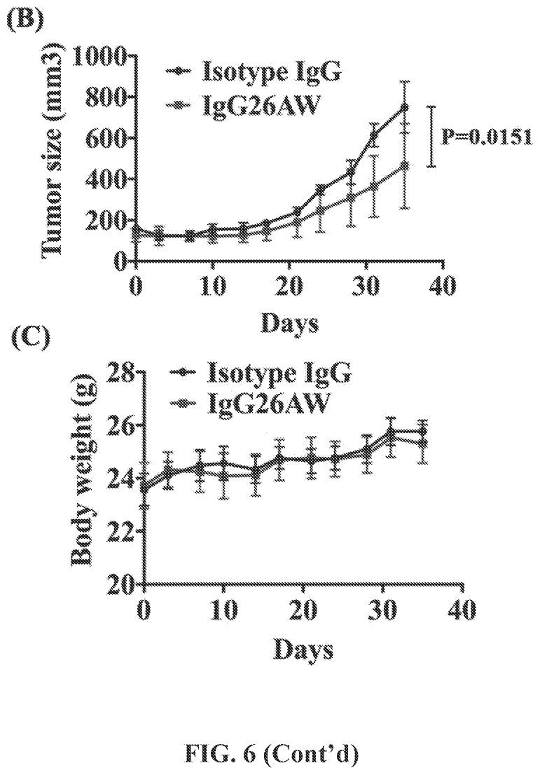

Chronic high-level expression of bioactive IL-1β is an important promotor of tumor development by driving the sustained NF-κB activation and mitogen activated protein kinase (MAPK) activity (FIG. 1, part A). In mice deficient in their own IL-1Ra and hence without a blocker of endogenous IL-1, tumor development was more rapid than that in wild type mice. For example, lung cancer patients had elevated levels of high sensitivity CRP and IL-6, indicating that the development of lung cancer is based on chronic lung inflammation, and this inflammation might be suppressed or reduced by IL-1β blockade. Additionally, in Ridker et al.'s CANTOS (canakinumab anti-inflammatory Thrombosis Outcomes Study) studies (N Engl J Med 2017; 377:1119-31), by using an IL-1β-neutralizing antibody for the prevention of reoccurrence of cardiovascular events reduced lung cancer incidence and mortality. This study was used to prove the importance of inflammation on the progression and development of lung cancer. It is also known that epithelial cells A549 are capable of secreting chemo-attractants and proinflammatory cytokines, which are important mediators in both lung defense and inflammation. Therefore, to determine whether blocking IL-1β signaling by IgG26AW suppresses human lung cancer growth, we utilized the A549 xenograft system in nude mice. First, to test both whether A549 cells respond to IL-1β stimulation and whether IgG26AW efficiently blocks the activation amplified by IL-1β, we used recombinant IL-1β to strongly induce the NF-κB pathway, which is critical for IL-1β-mediated downstream chain reactions, in A549 cells and treated them in parallel with IgG26AW and isotype IgG. As shown in FIG. 6, part A, IgG26AW efficiently blocked the p-JNK and p-p38 signals and the degradation of IκB-α even at very high doses of IL-1β (1000 pM) stimulation in A549 cells. Second, we treated A549 tumor-bearing nude mice with IgG26AW (10 μg/kg) three times a week for a total of 5 weeks. Excitingly, IgG26AW therapy reduced the tumor size from 730 mm³ to 480 mm³ without influencing the body weight (FIG. 6, parts B and C).

### Example 8: IgG26AW Inhibits Human Breast Cancer Progression and Metastasis

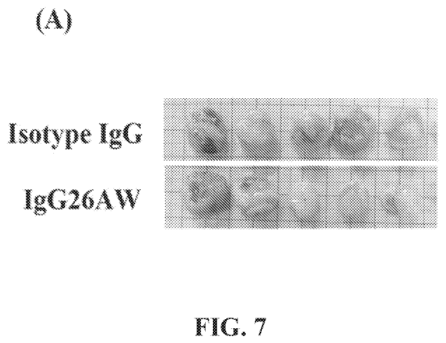

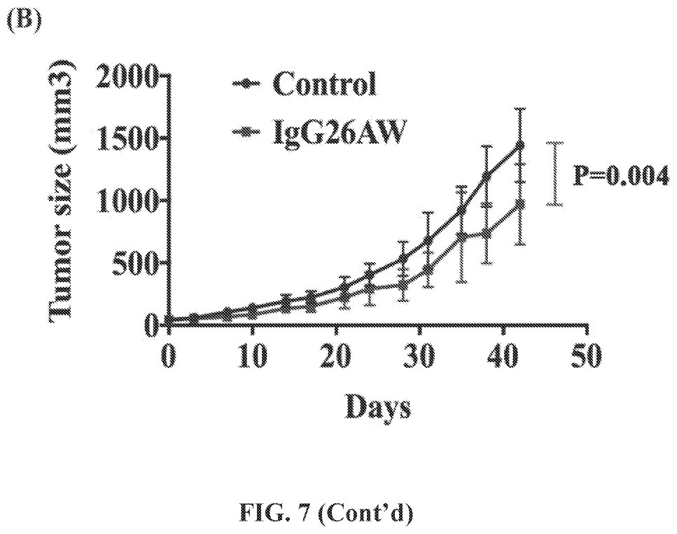

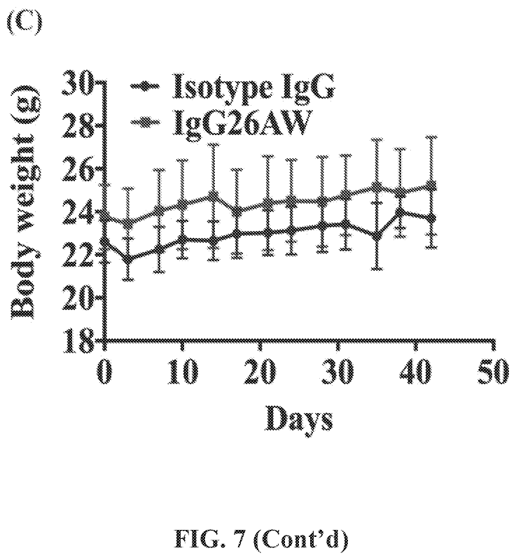

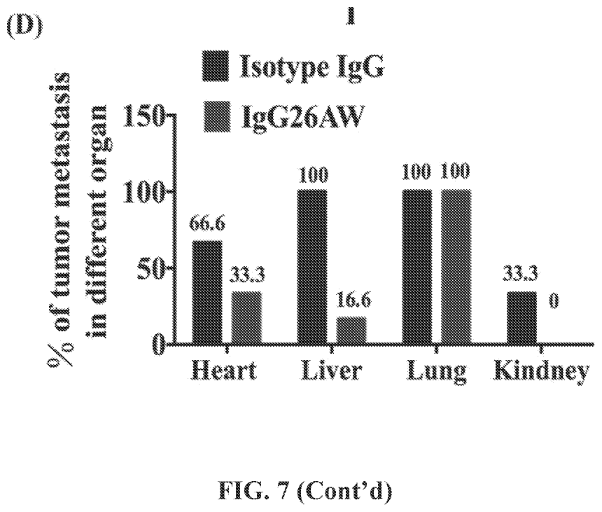

IL-1β expression is elevated in a variety of cancers (including breast, prostate, colon, and head and neck cancers and melanomas), and patients with IL-1β-producing tumors generally have a worse prognosis. In preliminary reports, endogenous IL-1β promotes metastasis of melanoma cells by upregulating tumor-cells that bind to endothelial cells via inducing adhesion molecules such as intracellular adhesion molecule-1 (ICAM-1) and vascular cell adhesion molecule-1 (VCAM-1); therefore, much attention has been paid to the role of IL-1β in metastasis. Holen et al. (Oncotarget 2016; 7:75571-84.) identified IL-1β as a potential prognostic biomarker for early breast cancer patients at increased risk for the subsequent development of skeletal metastases. MDAMB-231 cells are commonly used to model late-stage breast cancer. Since these cells lack the growth factor receptor HER2, they represent a good model of triple-negative breast cancer that displays the worst outcome of all breast cancer subtypes due to its propensity for early relapse and development of resistance to chemotherapeutic drugs. MDA-MB-231 cells are invasive in vitro; when implanted orthotopically, MDA-MB-231 cells produce xenografts that spontaneously metastasize to lymph nodes and other organs. Here, we utilized an orthotopic xenograft mouse model of human breast cancer cells to test whether IgG26AW treatment can delay tumor cell growth and even cancer metastasis. MDA-MB-231 cells were injected into the fat pads of immunodeficient ASID mice. When the tumor growth was up to 5 mm, tumor-bearing mice were treated with IgG26AW, canakinumab and isotype IgG via intravenous injection three times a week, and the tumor growth was measured. As shown in FIG. 7, part B, IgG26AW significantly suppressed the tumor growth of human breast cancer cells from 1420 mm³ to 950 mm³ without influencing the body weight (FIG. 7, parts A and C) in ASID mice. Remarkably, IgG26AW treatment significantly diminished the breast cancer metastasis process in the heart, liver, and kidney (FIG. 7, part D). In summary, systemic treatment with a neutralizing IL-1β-specific antibody mainly delayed the process of oncogenesis and metastasis. Some cancers spontaneously release IL-1β, as they can intrinsically activate both the expression of pro-IL-1β and the catalytic functions of caspase-129. Therefore, the blockade of IL-1β may constitute an important therapeutic rationale to impair tumor development and progression. Many human cancers are etiologically linked to chronic inflammatory processes. This is well documented for gastric, hepatic and colorectal cancers. Although IL-1β blockade does not directly kill cancer cells, it can be combined with other anti-cancer drugs to reduce therapeutic side effects and for palliative care.

### Example 9: PGG Synergistically Inhibits NF-κB Signaling with IgG26AW Antibody

1,2,3,4,6-Penta-O-Galloyl-β-D-Glucose (PGG) is a hydrolysable tannin and composed of five galloyl groups with a glucose at its core. PGG has been traditionally reported in plants that are commonly used in Chinese medicine. It belongs to the group of gallotannins. Many plants rich in PGG have been used ancestrally by the local communities of Africa, Asia, and Latin America in treatments against malaria, inflammation, snake and scorpion bite, diabetes, chronic diarrhea, toxicosis and microbial infections. PGG obtain more attention recently because of its therapeutic potentials (anti-inflammatory, anti-carcinogenic, antidiabetics and antioxidant). In previous studies revealed that PGG modulated the NF-κB and MAPK signaling pathways by altering genes and proteins expression that include septin-7, ataxin-2 and adenylosuccinate synthetase isozyme 2. These proteins were correlated with the neurodegenerative diseases and associated with the control of Alzheimer's disease pathogenesis. Additionally, PGG has therapeutic potential for the treatment of hepatocellular carcinoma, one of the most prevalent malignancies and deadliest cancers. In diabetes therapy, some reports indicated that single dose (10, 25, 50 and 100 mg/kg) of PGG isolated from mango leaves (Mangifera indica) dependently inhibited the 11f3-HSD-1 activity in liver and adipose tissue. PGG ameliorates high-fat diet induced diabetes in male C57BL/6 mice. Also, in vivo and in vitro studies show that PGG has an anti-inflammatory effect due to the inhibition of L-selection (CD62L) to treat against with atherosclerosis, colitis, and inflammatory skin damages.

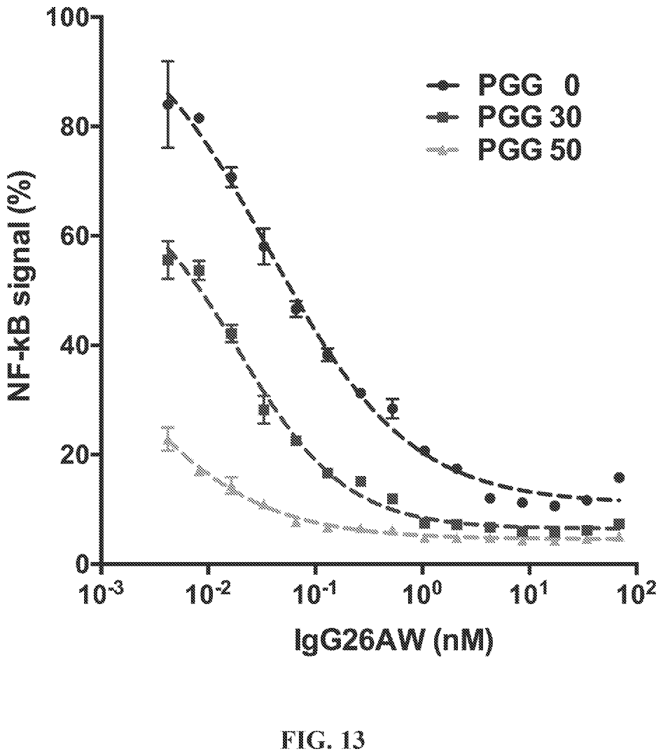

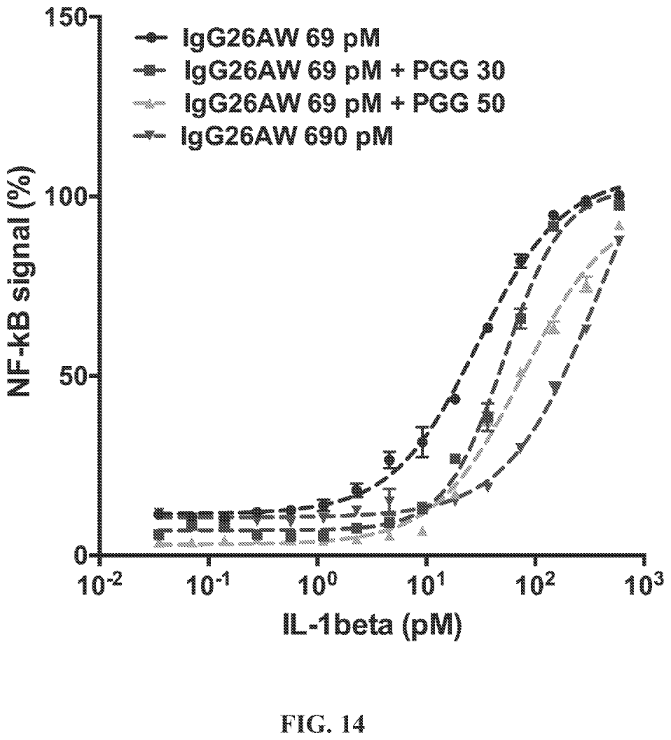

IgG26AW mainly inhibits the function of IL-1β, which is different from the wide-ranging effect of PGG. We combined PGG and IgG26AW simultaneously to test whether it can effectively inhibit the inflammation caused by IL-1β. As FIG. 13 shown, PGG synergistically reduce NF-kB signaling driven by IL-1β with IgG26AW. PGG can even suppress the remaining inflammatory response after IgG26AW strongest suppression. This result reveals that we can use a limited dose of antibody drugs together with broadly effective anti-inflammatory supplements such as PGG, which may reduce the side effects of high dose antibody therapy. We also test this possibility shown in FIG. 14. 69 pM IgG26AW combined with 50 μM PGG demonstrated that this combination efficiently inhibits the NF-κB signaling as similar as 690 pM IgG26AW can do. In conclusion, IgG26AW worked with PGG can synergistically diminish the inflammation response and reduce the usage amount of IgG26AW.

### Example 10: Discussion

Phage-displayed synthetic human antibody libraries can be used to decipher the natural antibody responses and to develop novel antibodies against diverse antigens. In this report, we used synthetic libraries to successfully identify the IL-1β antibody IgG26, which has an inhibitory effect on downstream IL-1β signaling. Although the affinity of the antibody was not good enough at first, the individual CDRs were re-examined through high-throughput screening of optimization libraries. Coupled with the protein X-ray crystal structure, we sped up the optimization process to achieve our desired goals; thus, the antibody has a specified binding region, an inhibitory or acceleratory ability or the destructive function for other associated protein binding. We have determined the protein structure of the 26-Fab/IL-1β bound states and have further demonstrated that the final version IgG26AW has a unique binding region on IL-1β that completely overlaps with IL-1RI, resulting in direct competition for IL-1β binding. The structure of the 26-Fab/IL-1β complex also showed that 26-Fab binding to IL-1β interfered with the critical region for IL-1RAcP binding. Compared to the mechanisms used in clinical practice for IL-1β blockade (blocking IL-1β binding to IL-1R or inhibiting recruitment of IL-1RAcP), IgG26AW simultaneously blocked the interactions of IL-1β with both IL-RI and IL-1RAcP to prevent the assembly of the IL-1β/IL-1RI/IL-1RAcP ternary complex. This explains why IgG26AW has a better neutralized ability compared with canakinumab and gevokizumab. The crystal structures of two therapeutic antibodies, canakinumab and gevokizumab, in complex with IL-1β have been reported 36. Two complex structures (PDB IDs: 4G6J and 4G6M) can be superimposed onto the IL-1β/IL-1RI/IL-1RAcP ternary complex to compare their IL-1β binding region with IgG26AW (FIG. 3, parts A and B). Canakinumab and IL-1RI share a small overlapping region on IL-1β, and this explains the receptor-blocking mechanism. Relatively, gevokizumab interacted with the IL-1β region and did not overlap with either receptor binding site. We have no way to speculate the possible mechanism from the gevokizumab structure. However, the fact is that gevokizumab decreases the association rate for the binding of IL-1β to its receptor and inhibits the subsequent recruitment of IL-1RAcP as indicated by a series of biophysical experiments. Therefore, different antibodies binding to different regions of the antigen will cause different physiological effects.

In addition, following administration, the in vivo binding of therapeutic IgG to circulating IL-1β results in the formation of an IgG-IL-1β complex. This complex, due to its larger molecular size, is expected to be eliminated at a much slower rate than the free IL-1β, thus resulting in the elevation of total IL-1β levels in human serum. Therefore, in our case, IL-1β bound by IgG26AW may not respond with IL-1RI again in the human serum because the paratope of IL-1β bound by IL-1RI is completely hidden after binding to IgG26AW. Compared with gevokizumab, the IL-1β molecule still has binding activity with IL-1RI in the gevokizumab/IL-1β complex due to incomplete suppression of downstream signaling.

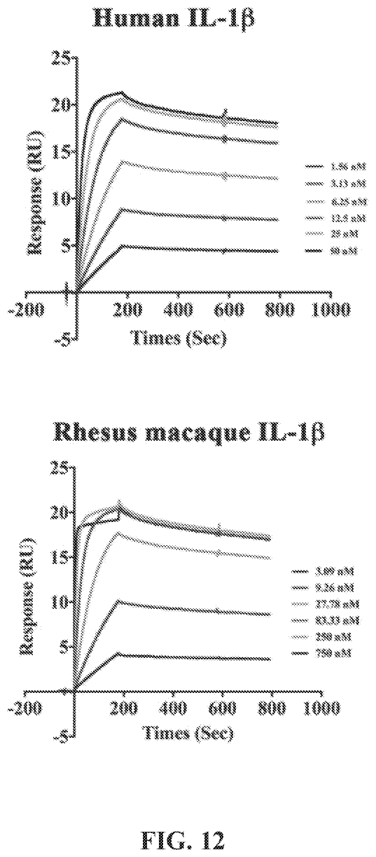

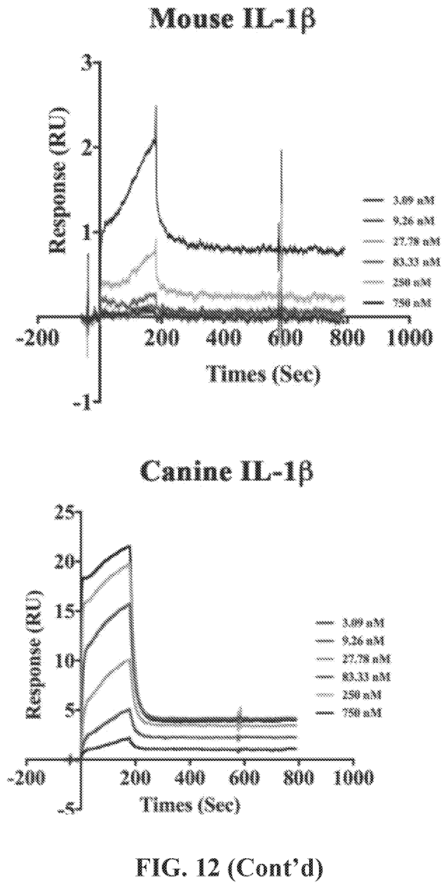

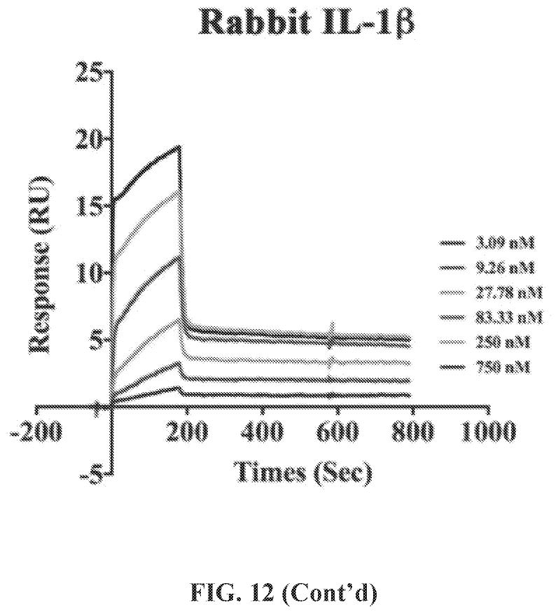

We also compared the neutralized ability between IgG26AW and canakinumab in an in vivo cytokine biomarker assay. IgG26AW had a more efficient inhibitory effect on inducing the mouse IL-6 than canakinumab at the 0.2 mg/kg dosage. This means that we can treat patients with a lower dosage of antibodies, which can reduce costs, side effects, resistant responses or antidrug antibodies (ADAs). IgG26AW has the potential to be developed into a therapeutic antibody. Ideally, we should validate the neutralized function of IgG26AW in a mouse disease model, but unfortunately, IgG26AW did not recognize mouse IL-1β (Table 4 and FIG. 12). Therefore, we chose two different human cell types (lung and breast cancers) from the xenograft tumor model to preliminarily test the anti-IL-1β blockade strategy in cancer treatments. The IgG26AW antibody as the therapeutic drug was injected into two xenograft mouse models. Although the IgG26AW treatment did not completely eliminate the cancers, we observed that the treatment of IgG26AW partially inhibited the growth and metastasis of tumors in lung and breast cancer models. In previous studies, anti-VEGF therapy led to a reduction in macrophage infiltration in MDA-MB-231 xenograft models. Roland's group found that the inhibition of VEGF receptor activation resulted in changes in intratumoral levels of IL-1β and CXCL1 that correlate with changes in immune cell infiltration (PLoS One 2009; 4:e7669). Subsequently, serum levels of IL-1β and IL-6 correlate with tumor response to anti-VEGF therapy and may be predictive clinical markers. Moreover, IL-1β produced by an aggressive breast cancer cell line, MDA-MB-231, is one of the factors that dictates their interactions with mesenchymal stem cells through chemokine production. Recently, chemokines and chemokine receptors have also been proven to be activators by promoting the initiation or progression of cancers. When they reduce the secretion of IL-1β by the shRNA approach in MDA-MB-231 cells, the cells reduced their motility in the presence of medium from MSCs cells that had been in contact with shIL-1β MDA-MB-231 cells. These data indicated that metastatic breast cancer cells can stimulate their microenvironment to produce IL-1β and other undefined factors activate the NF-κB pathway and stimulate the production of chemokines by MSC, which in turn will increase the aggressiveness of the breast cancer cells. Therefore, combined with our findings, it is known that (1) inhibition of the action of IL-1β spontaneously produced by tumor cells may suppress tumor growth in vivo; (2) inflammation is positively correlated with the incidence of tumor growth; and (3) IgG26AW treatment is functional and gainful for the inhibition of cancer growth. In addition, the best therapeutic approach for cancers is likely to be a combination of anticancer drugs worked by different mechanisms, thereby increasing the likelihood of a cure for cancer while reducing the side effects of cancer drugs. For example, the combination of anti-VEGF therapy with anti-IL-1β therapy, which simultaneously blocks the VEGF-induced NF-κB pathway and the IL-1β-induced amplification loop, may more strongly benefit triple negative breast cancer patients than single-drug treatment.

The main function of antibodies is to recognize foreign antigens and to help to launch the adaptive immune response in general physical conditions. This purpose is completely different from using antibodies as therapeutic reagents (passive immune) to cure human diseases. Accordingly, consideration for the therapeutic use of antibodies depends not only on the high binding specificity but also on the overall balanced results we will obtain in its final physiological activity. Each drug has its own strengths and weaknesses; moreover, each patient has a different genetic background that responds differently to the same drug. Therefore, we must develop different kinds of drugs for the same target. Multiple choices of drugs can promote the development of precision medicine. For example, personalizing precision medicine with combination therapies improves outcomes in cancer. Therefore, IgG26AW is a new candidate IL-1β inhibitor for adjunctive therapy to treat inflammation-related diseases or cancers in which the role of IL-1β is critical to pathogenesis.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An isolated antibody, which binds to Interleukin-1β (IL-1β), wherein the antibody comprises:
(a) a heavy chain variable domain (V_{H}), which comprises
(i) a heavy chain complementary determining region 2 (HC CDR2) set forth as WPX₁X₂GX₃TY or WPX₁GX₃TY, in which X₁, X₂ or X₃ is selected from any one of amino acids, and
(ii) a heavy chain complementary determining region 3 (HC CDR3) comprising NGYWNYI, AGHHTGA, ALKPTSA, DSRKPRAM, GPGHTNA, or ETNPIQA; and
(b) a light chain variable domain (V_{L}), which comprises
(i) a light chain complementary determining region 1 (LC CDR1) set forth as X₄X₅G, in which X₄ or X₅ is selected from any one of amino acids, and
(ii) a light chain complementary determining region 3 (LC CDR3) comprising YSNFPI.

2. The isolated antibody of claim 1, wherein the heavy chain variable domain (V_{H}) further comprises a heavy chain complementary determining region 1 (HC CDR1) comprising VDMA, KDNA, KDMA, DHNA, SHMA, DNAA, or NGYS.

3. The isolated antibody of claim 1, wherein the light chain variable domain (V_{L}) further comprises a light chain complementary determining region 2 (LC CDR2) comprising YSTAS, SQSTD, or HTSRS.

4. The isolated antibody of claim 3, wherein the antibody (IgG26) comprises:
a HC CDR1 of amino acids consisting of NGYS;
a HC CDR2 of amino acids consisting of WPYGGFTY;
a HC CDR3 of amino acids consisting of NGYWNYI;
a LC CDR1 of amino acids consisting of SWG;
a LC CDR2 of amino acids consisting of YSTAS; and
a LC CDR3 of amino acids consisting of YSNFPI.

5. The isolated antibody of claim 3, wherein the antibody (IgGF4) comprises:
a HC CDR1 of amino acids consisting of VDMA;
a HC CDR2 of amino acids consisting of WPYGGFTY;
a HC CDR3 of amino acids consisting of NGYWNYI;
a LC CDR1 of amino acids consisting of SWG;
a LC CDR2 of amino acids consisting of HTSRS; and
a LC CDR3 of amino acids consisting of YSNFPI.

6. The isolated antibody of claim 3, wherein the antibody (IgG26A) comprises:
a HC CDR1 of amino acids consisting of KDMA;
a HC CDR2 of amino acids consisting of WPREGFTY;
a HC CDR3 of amino acids consisting of NGYWNYI;
a LC CDR1 of amino acids consisting of SWG;
a LC CDR2 of amino acids consisting of YSTAS; and
a LC CDR3 of amino acids consisting of YSNFPI.

7. The isolated antibody of claim 3, wherein the antibody (IgG26AW) comprises:
a HC CDR1 of amino acids consisting of VDMA;
a HC CDR2 of amino acids consisting of WPREGWTY;
a HC CDR3 of amino acids consisting of NGYWNYI;
a LC CDR1 of amino acids consisting of SWG;
a LC CDR2 of amino acids consisting of HTSRS; and
a LC CDR3 of amino acids consisting of YSNFPI.

8. The isolated antibody of claim 3, wherein the antibody comprises a V_{H} comprising the amino acid sequence of:
EVQLVESGGGLVQPGGSLRLSCAASGFTIVDMAIHWVRQAPGKGLEWVAR
IWPREGWTYYADSVKGRFTISADTSKNTAYLQMNSLRAEDTAVYYCARFN
GYWNYIMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKAEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKDYKCKVSNKALPAPIEKTISKAKGQPREPQ
VYTLPPSRDELTRNQVSLTCLVKGFYPSDIAVEWESNGQPENNYKTTPPV
LDSDGSFFLYSKLTVDKSRWQQGNVFSCSVMHEALHNHYTQKSLSLSPG
K,
and/or
a V_{L} comprising the amino acid sequence of:
DIQMTQSPSSLSASVGDRVTITCRASQDVSWGVAWYQQKPGKAPKLLIHT
SRSLYSGVPSRFSGSGSGTDFTLTISSLQPEDFATYYCQQYSNFPITFGD
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

9. The isolated antibody of claim 1, wherein the antibody specifically binds human IL-1β.

10. The isolated antibody of claim 1, wherein the antibody cross-reacts with human IL-1β and a non-human IL-1β.

11. The isolated antibody of claim 10, wherein the non-human IL-1β is a mouse IL-1β, rabbit IL-1β, monkey IL-1β, or canine IL-1β.

12. The isolated antibody of claim 8, wherein the antibody comprises a heavy chain variable domain (V_{H}) that is at least 80%, preferably 85%, more preferably 90%, identical to the heavy chain variable domain of antibody IgG26, IgGF4, IgG26A or IgG26AW, and a light chain variable domain (V_{L}) that is at least 80%, preferably 85%, more preferably 90%, identical to the light chain variable domain of antibody IgG26, IgGF4, IgG26A or IgG26AW.

13. The isolated antibody of claim 1, wherein the antibody is a human antibody or a humanized antibody.

14. The isolated antibody of claim 1, wherein the antibody is a full-length antibody or an antigen binding fragment thereof.

15. The isolated antibody of claim 14, wherein the antibody is a full-length antibody, which is an IgG molecule.

16. The isolated antibody of claim 1, wherein the antibody is further conjugated to a detectable label, an immune adhesion molecule, an imaging agent, a therapeutic agent, or a cytotoxic agent.

17. The isolated antibody of claim 16, wherein said imaging agent is selected from the group consisting of: a radiolabel, an enzyme, a fluorescent label, a luminescent label, a bioluminescent label, a magnetic label, and biotin.

18. The isolated antibody of claim 16, wherein said therapeutic or cytotoxic agent is selected from the group consisting of: an anti-metabolite, an alkylating agent, an antibiotic, a growth factor, a cytokine, an anti-angiogenic agent, an anti-mitotic agent, an anthracycline, toxin, and an apoptotic agent.

19. A pharmaceutical composition comprising an isolated antibody of claim 1, and a pharmaceutically acceptable carrier.

20. The pharmaceutical composition of claim 19, wherein the pharmaceutically acceptable carrier comprises a buffering agent, a surfactant, a salt, an amino acid, an antioxidant, a sugar derivative, or a combination thereof.

21. The pharmaceutical composition of claim 20, wherein the sugar derivative is a non-reducing sugar, a sugar alcohol, a polyol, a disaccharide, or a polysaccharide.

22. The pharmaceutical composition of claim 19, further comprising 1,2,3,4,6-Penta-O-Galloyl-β-D-Glucose (PGG).

23. The pharmaceutical composition of claim 22, wherein a concentration of the PGG ranges from 1-500 μM.

24. The pharmaceutical composition of claim 22, wherein a concentration of the isolated antibody ranges from 1 pM-1000 nM.

25. A nucleic acid or a nucleic acid set, which collectively encode the isolated antibody set forth in claim 1.

26. The nucleic acid or nucleic acid set of claim 25, wherein the nucleic acid or nucleic acid set is a vector or a vector set.

27. A host cell, comprising the vector or vector set of claim 26.

28. The host cell of claim 27, which is selected from the group consisting of a bacterial cell, a yeast cell, an insect cell, a plant cell, and a mammalian cell.

29. A method for producing an antibody binding to human IL-1β, the method comprising:
(i) culturing the host cell of claim 27 under conditions allowing for expression of the antibody that binds human IL-1β; and
(ii) harvesting the cultured host cell or culture medium for collection of the antibody that binds human IL-1β.

30. The method of claim 29, further comprising purifying the antibody that binds human IL-1β.

31. A method for treating IL-1β mediated disease in a subject, the method comprising administering to a subject in need thereof an effective amount of the antibody of claim 1.

32. The method of claim 31, wherein the subject is a human patient having, suspected of having, or at risk for the IL-1β mediated disease.

33. The method of claim 32, wherein the IL-1β mediated disease is an inflammatory disease, an autoimmune disease, or a cancer.

34. The method of claim 33, wherein the disease is an autoimmune disease comprising cryopyrin-associated periodic syndrome, neonatal-onset multisystem inflammatory disease, rheumatoid arthritis, juvenile rheumatoid arthritis, spondyloarthropathy, ankylosing spondylitis, multiple sclerosis, psoriasis, plaque psoriasis, gouty arthritis, osteoarthritis, or Kawasaki disease.

35. The method of claim 33, wherein the IL-1β mediated disease is an inflammatory disease comprising Kawasaki disease, chimeric antigen receptor T cell (CAR-T) induced cytokine release syndrome, CAR-T-induced related encephalopathy, diffuse parenchymal lung disease (DPLD), chronic obstructive pulmonary disease (COPD), aortic aneurysm, neuropathic pain, or graft-versus-host disease (GVHD).

36. The method of claim 33, wherein the IL-1β mediated disease is a cancer comprising leukemia, gastric carcinoma, adenocarcinoma, mesothelioma, lung cancer, breast cancer, prostate cancer, colon cancer, head and neck cancer, melanoma, pancreatic ductal adenocarcinoma, colorectal cancer (CAC), or hypereosinophilic syndrome (HES).

37. The method of claim 36, wherein the leukemia comprising juvenile myelomonocyte leukemia (JMML), chronic myelomonocytic leukemia (CMML) or chronic eosinophilic leukemia.

38. The method of claim 32, wherein the IL-1β mediated disease comprises gout, type II diabetes mellitus, or amyotrophic lateral sclerosis.

39. The method of claim 31, wherein the subject has undergone or is undergoing an additional treatment of the IL-1β mediated disease.

40. A method for detecting presence of IL-1β, the method comprising:
(i) contacting a biological sample suspected of containing IL-1β with the isolated antibody of claim 1, and
(ii) measuring binding of the antibody to IL-1β in the sample.

41. The method of claim 40, wherein the biological sample is obtained from a human subject suspected of having or at risk for a disease associated with IL-1β.

42. The method of claim 40, wherein the contacting step is performed by administering the subject an effective amount of the anti-IL-1β antibody.


SEQUENCE LISTING (34 sequences)

SEQ ID NO: 1 - protein, 8 aa, Artificial Sequence
  HC CDR2-1
  MISC_FEATURE (3)..(4): Xaa is any one of amino acids
  MISC_FEATURE (6)..(6): Xaa is any one of amino acids
WPXXGXTY

SEQ ID NO: 2 - protein, 7 aa, Artificial Sequence
  HC CDR2-2
  MISC_FEATURE (3)..(3): Xaa is any one of amino acids
  MISC_FEATURE (5)..(5): Xaa is any one of amino acids
WPXGXTY

SEQ ID NO: 3 - protein, 7 aa, Artificial Sequence
  HC CDR3-1
NGYWNYI

SEQ ID NO: 4 - protein, 7 aa, Artificial Sequence
  HC CDR3-2
AGHHTGA

SEQ ID NO: 5 - protein, 7 aa, Artificial Sequence
  HC CDR3-3
ALKPTSA

SEQ ID NO: 6 - protein, 8 aa, Artificial Sequence
  HC CDR3-4
DSRKPRAM

SEQ ID NO: 7 - protein, 7 aa, Artificial Sequence
  HC CDR3-5
GPGHTNA

SEQ ID NO: 8 - protein, 7 aa, Artificial Sequence
  HC CDR3-6
ETNPIQA

SEQ ID NO: 9 - protein, 3 aa, Artificial Sequence
  LC CDR1
  MISC_FEATURE (1)..(2): Xaa is any one of amino acids
XXG

SEQ ID NO: 10 - protein, 6 aa, Artificial Sequence
  LC CDR3
YSNFPI

SEQ ID NO: 11 - protein, 4 aa, Artificial Sequence
  HC CDR1-1
VDMA

SEQ ID NO: 12 - protein, 4 aa, Artificial Sequence
  HC CDR1-2
KDNA

SEQ ID NO: 13 - protein, 4 aa, Artificial Sequence
  HC CDR1-3
KDMA

SEQ ID NO: 14 - protein, 4 aa, Artificial Sequence
  HC CDR1-4
DHNA

SEQ ID NO: 15 - protein, 4 aa, Artificial Sequence
  HC CDR1-5
SHMA

SEQ ID NO: 16 - protein, 4 aa, Artificial Sequence
  HC CDR1-6
DNAA

SEQ ID NO: 17 - protein, 4 aa, Artificial Sequence
  HC CDR1-7
NGYS

SEQ ID NO: 18 - protein, 5 aa, Artificial Sequence
  LC CDR2-1
YSTAS

SEQ ID NO: 19 - protein, 5 aa, Artificial Sequence
  LC CDR2-2
SQSTD

SEQ ID NO: 20 - protein, 5 aa, Artificial Sequence
  LC CDR2-3
HTSRS

SEQ ID NO: 21 - protein, 450 aa, Artificial Sequence
  IgG26AW VH
EVQLVESGGGLVQPGGSLRLSCAASGFTIVDMAIHWVRQAPGKGLEWVARIWPREGWTYYADSVKGRFTISADTSKNTAYLQMNSLRAEDTAVYYCARFNGYWNYIMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPIEKTISKAKGQPREPQVYTLPPSRDELTRNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, Artificial Sequence
  IgG26AW VL
DIQMTQSPSSLSASVGDRVTITCRASQDVSWGVAWYQQKPGKAPKLLIHTSRSLYSGVPSRFSGSGSGTDFTLTISSLQPEDFATYYCQQYSNFPITFGDGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 8 aa, Artificial Sequence
  IgG26AW VH CDR2
WPREGWTY

SEQ ID NO: 24 - protein, 3 aa, Artificial Sequence
  IgG26AW VL CDR1
SWG

SEQ ID NO: 25 - DNA, 59 bp, Artificial Sequence
  VL-F-KpnI
caggtgcacgatgtgatggtaccgatattcaaatgacccagagcccgagcagcctgagc

SEQ ID NO: 26 - DNA, 36 bp, Artificial Sequence
  VL-R
tgcagccaccgtacgtttgatttccaccttggtgcc

SEQ ID NO: 27 - DNA, 36 bp, Artificial Sequence
  VH-F
cgtgtcgcatctgaagtgcagctggtggaatcggga

SEQ ID NO: 28 - DNA, 48 bp, Artificial Sequence
  VH-R-NheI
gaccgatgggcccttggtgctagccgagctcacggtaacaagggtgcc

SEQ ID NO: 29 - DNA, 39 bp, Artificial Sequence
  IgG-Linker-F
aaggtggaaatcaaacgtacggtggctgcaccatctgtc

SEQ ID NO: 30 - DNA, 33 bp, Artificial Sequence
  IgG-Linker-R
ctgcacttcagatgcgacacgcgtagcaacagc

SEQ ID NO: 31 - protein, 8 aa, Artificial Sequence
  IgG26 H-CDR2
WPYGGFTY

SEQ ID NO: 32 - protein, 8 aa, Artificial Sequence
  IgG26A H-CDR2
WPREGFTY

SEQ ID NO: 33 - protein, 3 aa, Artificial Sequence
  IgGL1-1 L-CDR1
AGG

SEQ ID NO: 34 - protein, 3 aa, Artificial Sequence
  IgGL1-2 L-CDR1
RQG